(19) United States
(12) Patent Application Publication
(10) Pub. No.: US 2024/0125809 A1
(43) Pub. Date: Apr. 18, 2024

(54) AUTOMATIC EXTRACTION DEVICE AND USE METHOD THEREFOR

(71) Applicant: NANJING GENSCRIPT BIOTECH CO., LTD., Nanjing (CN)

(72) Inventors: Hao ZHANG, Nanjing (CN); Tao BAI, Nanjing (CN); Zhenbo SUN, Nanjing (CN); Jinwen WEI, Nanjing (CN); Yu FANG, Nanjing (CN); Liwei SUN, Nanjing (CN); Yuehua QIAO, Nanjing (CN); Zhun ZHANG, Nanjing (CN)

(73) Assignee: NANJING GENSCRIPT BIOTECH CO., LTD., Nanjing, Jiangsu (CN)

(21) Appl. No.: 18/396,656

(22) Filed: Dec. 26, 2023

Related U.S. Application Data
(63) Continuation of application No. PCT/CN2022/105856, filed on Jul. 15, 2022.

(30) Foreign Application Priority Data
Jul. 15, 2021 (CN) 202110801973.5

(51) Int. Cl. G01N 35/00; C12N 15/10; G01N 35/10

(52) U.S. Cl. CPC G01N 35/0098; C12N 15/1013; G01N 35/1072; G01N 2035/00564

(57) ABSTRACT

An automatic extraction device and a use method therefor. The automatic extraction device comprises a sample processing apparatus (100), a portal frame (200), a consumable assembly (300), and a pipetting apparatus (400). The sample processing apparatus (100) comprises a uniform mixing mechanism (130) and a magnetic attraction mechanism (140), which are respectively used for performing uniform mixing processing and magnetic attraction processing on a biological sample. The portal frame (200) comprises a base (201) and a moving beam (202) capable of moving relative to the base (201). The sample processing apparatus (100) and the consumable assembly (300) are provided on the base (201). The pipetting apparatus (400) is provided on the moving beam (202) for implementing a pipetting operation between the consumable assembly (300) and the sample processing apparatus (100). The automatic extraction device can be used for extracting and purifying a target biological substance, such that mechanical automation of an extraction or purification process can be achieved, thereby reducing the time and energy of operators while improving the extraction and purification efficiency.

### CROSS-REFERENCE TO RELATED APPLICATIONS

This application is a continuation of International Application No. PCT/CN2022/105856, filed on Jul. 15, 2022, which claims priority to Chinese Application No. 202110801973.5, filed on Jul. 15, 2021, the entire contents of each of which are incorporated herein by reference.

### TECHNICAL FIELD

This specification relates to the field of biological instruments and apparatuses, and in particular to an automatic extraction device and a use method therefor.

### BACKGROUND

In modern bioengineering, the extraction or purification processes of target biological substances (such as plasmids) are typically carried out by combining manual and mechanical methods, but the manual operation part accounts for a high proportion. Not only it is required to select an appropriate extraction or purification process, but also the workload is large and takes a long time. Moreover, manual operations often cause individual differences, which would affect the accuracy of results, and the consistency of process execution is not ideal. When there are a large number of samples, the throughput of detection is also limited, and it is difficult to meet the demand for extracting or purifying high-throughput samples due to the current rapid development of bioengineering.

Therefore, it is necessary to provide an automatic extraction device and a use method therefor, so as to enable mechanical automation of an extraction or purification process of a target biological substance.

### SUMMARY

One of embodiments of this specification provides an automatic extraction device. The automatic extraction device is configured to extract a target biological substance. The automatic extraction device comprises a sample processing apparatus, a consumable assembly, a pipetting apparatus and a portal frame. the sample processing apparatus comprises a uniform mixing mechanism and a magnetic attraction mechanism, which are respectively configured to perform uniform mixing processing and magnetic attraction processing on a biological sample. The portal frame comprises a base and a moving beam capable of moving relative to the base. The sample processing apparatus and the consumable assembly are provided on the base, and the pipetting apparatus is provided on the moving beam. The pipetting apparatus is configured to be capable of implementing a pipetting operation between the consumable assembly and the sample processing apparatus.

One of the embodiments of this specification provides a use method for the automatic extraction device as described above. The use method comprises controlling the pipetting apparatus to add a first reagent and a first magnetic bead of the consumable assembly into the sample tube of the sample processing apparatus. A biological sample is contained in the sample tube. The use method comprises controlling the uniform mixing mechanism to drive the sample tube for uniform mixing processing. The use method further comprises controlling the magnetic attraction mechanism to perform magnetic attraction processing on a uniformly mixed liquid in the sample tube. The use method further comprises, after standing for a first preset time, controlling the pipetting apparatus to collect a first supernatant from the sample tube, or to remove the first supernatant from the sample tube while retaining the attracted first magnetic bead.

### BRIEF DESCRIPTION OF THE DRAWINGS

This specification will be further illustrated by way of exemplary embodiments, and these exemplary embodiments will be described in detail with reference to the accompanying drawings. These embodiments are not restrictive, and in these embodiments, the same numerals represent the same structures, in which:

FIG. 1 is a schematic structural diagram of an automatic extraction device according to some embodiments of this specification from a first perspective;

FIG. 2 is a schematic structural diagram of an automatic extraction device according to some embodiments of this specification from a second perspective;

FIG. 3 is a schematic structural diagram of an automatic extraction device according to some embodiments of this specification from a third perspective;

FIG. 4 is a schematic structural diagram of a consumable assembly according to some embodiments of this specification;

FIG. 5 is a schematic structural diagram of a reagent kit assembly according to some embodiments of this specification;

FIG. 6 is a schematic structural diagram of a reagent kit according to some embodiments of this specification;

FIG. 7 is a schematic structural diagram of a pipette tip box according to some embodiments of this specification;

FIG. 8 is a schematic diagram of an internal structure of a pipette tip box according to some embodiments of this specification;

FIG. 9 is a schematic structural diagram of a pipetting apparatus according to some embodiments of this specification;

FIG. 10 is a schematic structural diagram of a sample processing apparatus according to some embodiments of this specification;

FIG. 11 is a schematic structural diagram of a uniform mixing mechanism according to some embodiments of this specification;

FIG. 12 is a schematic structural diagram of a tube holder according to some embodiments of this specification;

FIG. 13 is a schematic structural diagram of a magnetic attraction mechanism according to some embodiments of this specification;

FIG. 14 is a schematic structural diagram of a magnetic attraction mechanism according to some other embodiments of this specification;

FIG. 15 is a schematic structural diagram of the arrangement of magnets of a magnetic attraction mechanism according to some embodiments of this specification;

FIG. 16 is a demonstration diagram of magnetic attraction of a magnetic attraction mechanism according to some embodiments of this specification;

FIG. 17 is a demonstration diagram of magnetic attraction of a magnetic attraction mechanism according to some embodiments of this specification; and

FIG. 18 is a schematic diagram of a plurality of extraction channels of an automatic extraction device according to some embodiments of this specification.

Reference signs: 100—Sample processing apparatus, 110—Sample tube, 120—Reaction tube, 130—Uniform mixing mechanism, 131—Uniform mixing driving motor, 132—Tube holder, 1321—Notch, 133—Position monitoring component, 1331—Photoelectric sensor, 1332—Baffle, 140—Magnetic attraction mechanism, 141—Magnet, 1411—Magnetic attraction bracket, 142—First magnetic attraction mechanism, 1421—First magnet, 1421a—Upper-layer sub-magnet, 1421b—Lower-layer sub-magnet, 1422—First magnetic attraction driving motor, 1423—Magnetic shield, 1424—Second magnetic attraction driving motor, 1425—First magnetic attraction bracket, 1426—Two linear guide rails, 1427—Lead screw, 143—Second magnetic attraction mechanism, 1431—Second magnet, 1432—Third magnetic attraction driving motor, 1433—Fourth magnetic attraction driving motor, 1434—Second magnetic attraction bracket, 1435—Guiding shaft, 150—Support structure, 200—Portal frame, 201—Base, 202—Moving beam, 203—Moving beam driving motor, 204—Moving beam guide rail, 205—Moving beam lead screw-nut pair, 206—First synchronous pulley assembly, 300—Consumable assembly, 301—Reagent kit assembly, 3011—Reagent kit, 3012—Sealing layer, 3013—Cavity, 302—Pipette tip box, 3021—Pipette tip, 3022—Box body, 3027—Accommodating cavity, 3023—Cover plate, 3024—Partition, 3025—Through hole, 3026—Target biological substance collection tube, 303—Reagent kit holder, 3031—Handle, 304—Consumable supporting seat, 305—Consumable assembly driving motor, 306—Consumable assembly guide rail, 400—Pipetting apparatus, 401—Pipette, 4011—Piston, 4012—Suction head, 402—Piston driving motor, 403—Driving plate, 404—Support plate, 405—Piston guide rail, 406—Piercing mechanism, 407—Spring, 408—Push plate, 409—Push rod, 410—Pipetting apparatus driving motor, 411—Pipetting apparatus guide rail, 412—Pipette lead screw-nut pair, 413—Second synchronous pulley assembly, 500—Waste liquid apparatus, 501—Waste liquid box, 502—Waste liquid box holder, 503—Waste liquid box cover plate through hole, 600—Coordinate system.

### DETAILED DESCRIPTION

For a clearer description of the technical solutions in the embodiments of this specification, the accompanying drawings required for describing the embodiments will be briefly described below. Apparently, the accompanying drawings in the following description show merely some examples or embodiments of this specification, and those of ordinary skill in the art may still apply this specification to other similar scenarios according to these accompanying drawings without any creative effort. Unless obvious from the linguistic context or otherwise stated, the same reference signs in the drawings represent the same structure or operation.

The terms “first”, “second” and the like used in this specification and the claims do not denote any sequence, quantity, or importance, but are only used to distinguish different constituent parts. Likewise, the terms “a”, “an” or the like also do not denote a quantity limitation, but mean that there is at least one. Unless otherwise stated, the terms “front”, “rear”, “lower”, “upper” and/or the like are merely for ease of description and are not intended to define one position or one spatial orientation. Generally, the terms “including” and “comprising” only imply the inclusion of explicitly identified steps and elements, and these steps or elements do not constitute an exclusive list. A method or a device may further comprise other steps or elements.

An automatic extraction device provided in some embodiments of the present application can be used for automatically extract and/or purify a target biological substance in a biological sample, and can greatly reduce the workload of operators (such as laboratory personnel or workers) and shorten the work time. The device adopts mechanical automatic operation in an entire process, and has high repeatability and high accuracy of results. In some embodiments, the automatic extraction device can be used for high-throughput sample processing, and significantly improve the efficiency of extraction and/or purification when processing a plurality of samples simultaneously. The target biological substance here refers to a substance to be extracted (or to be separated) and/or to be purified in the biological sample. In some embodiments, the target biological substance comprises plasmids (e.g., DNA plasmids, or RNA plasmids), proteins, nucleic acids, and any other feasible target biological substances. For example, the automatic extraction device can be used for automatically extracting and/or purifying plasmids. For another example, the automatic extraction device can be used for automatically extracting and/or purifying proteins. For still another example, the automatic extraction device can be used for automatically extracting and/or purifying nucleic acids. In some embodiments, the biological sample may be microorganisms (including bacteria, viruses, fungi, etc., such as Escherichia coli), blood, saliva, an animal tissue, a plant, food, or any other feasible biological samples. It should be understood that application scenarios of the automatic extraction device in the specification of the present application are merely some examples or embodiments of the present application. Those of ordinary skill in the art may still apply the present application to other similar scenarios according to these accompanying drawings without any creative effort.

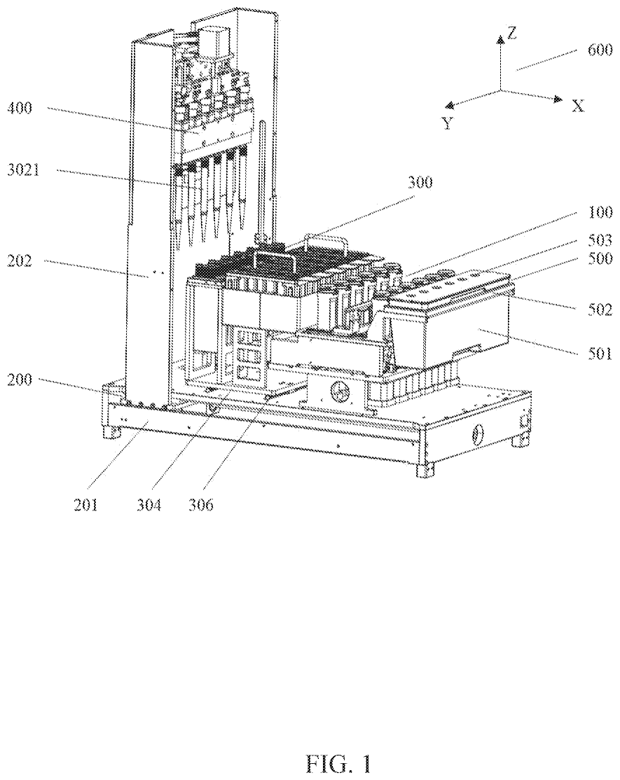

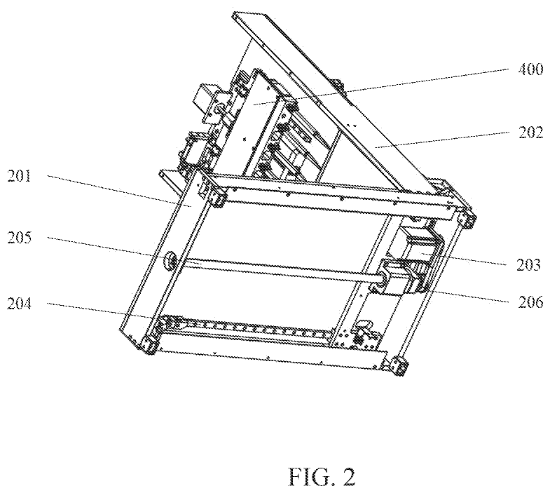

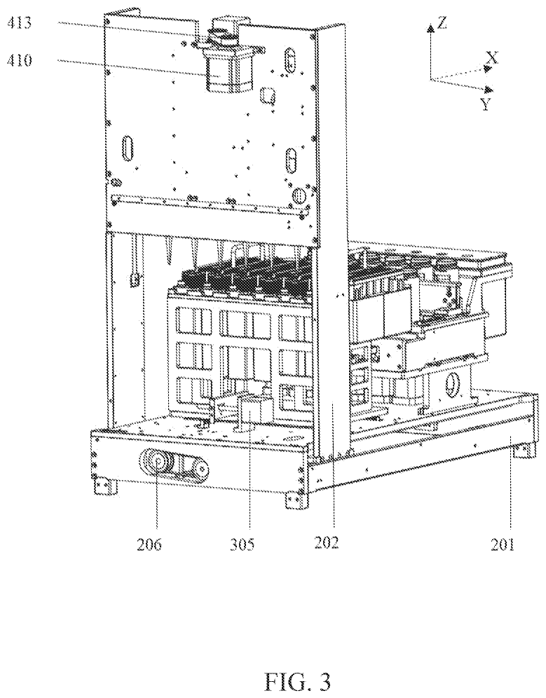

FIG. 1 is a schematic structural diagram of an automatic extraction device according to some embodiments of this specification from a first perspective; FIG. 2 is a schematic structural diagram of an automatic extraction device according to some embodiments of this specification from a second perspective; and FIG. 3 is a schematic structural diagram of an automatic extraction device according to some embodiments of this specification from a third perspective. It should be noted that the structure of the automatic extraction device shown in FIGS. 1 to 3 is merely for illustrative purposes and is not intended to limit the scope of the present application.

As shown in FIGS. 1 and 2, the automatic extraction device comprises a portal frame 200, a sample processing apparatus 100, a consumable assembly 300, a pipetting apparatus 400 and a waste liquid apparatus 500. It should be noted that the automatic extraction device may not be provided with the waste liquid apparatus 500 according to different requirements.

For ease of description, a coordinate system 600 as shown in FIG. 1 is used to further describe various assemblies and/or apparatuses of the automatic extraction device. The coordinate system 600 (e.g., a Cartesian coordinate system) comprises an X-axis, a Y-axis, and a Z-axis. The Z-axis is parallel to a vertical direction (for example, being perpendicular to the ground), the X-axis and the Y-axis are parallel to a horizontal direction (for example, being parallel to the ground), and the X-axis is perpendicular to the Y-axis. The Y-axis direction is also referred to as a first direction. The X-axis direction is also referred to as a second direction. The Z-axis direction is also referred to as a third direction.

The portal frame 200 is configured to support one or more assemblies and/or apparatuses (e.g., the sample processing apparatus 100, the consumable assembly 300, the pipetting apparatus 400, and/or the waste liquid apparatus 500) of the automatic extraction device. In some embodiments, the portal frame 200 comprises a base 201 and a moving beam 202. The sample processing apparatus 100, the consumable assembly 300 and the waste liquid apparatus 500 are provided on the base 201. For example, the sample processing apparatus 100, the consumable assembly 300 and the waste liquid apparatus 500 may be sequentially arranged on the base 201 at intervals (e.g., in the X-axis direction (i.e., the second direction)). The interval mentioned here refers to a certain spacing. The arrangement order of the assemblies and/or the apparatuses is not limited here. The arrangement order may be as shown in FIG. 1 in which the sample processing apparatus 100 is arranged on the base 201 and located between the consumable assembly 300 and the pipetting apparatus 400, or may be arranged in any other order. For another example, the sample processing apparatus 100, the consumable assembly 300 and/or the waste liquid apparatus 500 are detachably mounted on the base 201. The pipetting apparatus 400 is provided on the moving beam 202. For example, the pipetting apparatus 400 is detachably mounted on the moving beam 202. For another example, the pipetting apparatus 400 is movably mounted on the moving beam 202. That is, the pipetting apparatus 400 can move relative to the moving beam 202 (e.g., move in the Z-axis direction) so as to achieve liquid aspiration or dispensing. In some embodiments, the moving beam 202 comprises a column arranged perpendicular to the base 201. For example, the base 201 is disposed parallel to the ground (e.g., parallel to a XY plane), and the column is disposed perpendicular to the ground (e.g., parallel to a YZ plane). In some embodiments, the moving beam 202 may be of a single-column structure (a structure comprising a single column) or a multi-column structure (e.g., a structure comprising multiple columns). For example, as shown in FIG. 1, the moving beam 202 may comprise two columns (i.e., a double-column structure) which are respectively provided in parallel on two sides of the base 201 in the Y-axis direction (i.e., the first direction). For another example, the moving beam 202 may comprise one column (i.e., a single-column structure) which is provided on any side of the base 201 in the Y-axis direction (i.e., the first direction). The column may include a cylindrical column, a square column, etc. In some embodiments, when the moving beam 202 is of a multi-column structure, in addition to two parallelly arranged columns, the moving beam 202 is further provided with a cross beam perpendicular to the two columns (e.g., parallel to the Y-axis). Two ends of the cross beam are securely connected to the two columns respectively, and such an arrangement is conductive to the structural stability of the moving beam 202.

In some embodiments, the moving beam 202 is movably mounted on the base 201, that is, the moving beam 202 can move relative to the base 201 (e.g., in the X-axis direction (i.e., the second direction)). As the moving beam 202 moves, the pipetting apparatus 400 can move to above a position where liquid aspiration or dispensing is required. In some embodiments, the moving beam 202 may be connected to the base 201 via a moving beam guide rail 204. The moving beam guide rail 204 may be a linear guide rail. The moving beam 202 may be in transmission connection with a moving beam driving motor 203. The moving beam 202 moves on the moving beam guide rail (e.g., in the X-axis direction (i.e., the second direction)) under the drive of the moving beam driving motor 203. In some embodiments, the moving beam 202 may be in transmission connection with the moving beam driving motor 203 by means of a moving beam lead screw-nut pair 205. For example, the moving beam 202 may be connected to the moving beam lead screw-nut pair 205, the moving beam lead screw-nut pair 205 may be connected to the moving beam driving motor 203 via a first synchronous pulley assembly 206, and the moving beam driving motor 203 rotates by means of the moving beam lead screw-nut pair 205, thereby driving the moving beam 202 to move in the X-axis direction (i.e., the second direction). In some embodiments, the moving beam guide rail is securely mounted on the base 201. For example, when the moving beam 202 is of a double-beam structure, the moving beam guide rails respectively corresponding to the two columns may be securely mounted on two sides of the base 201 in the Y-axis direction (i.e., the first direction), such that the two columns can be moved on the corresponding moving beam guide rails. For another example, when the moving beam 202 is of a column structure, the moving beam guide rail may be securely mounted on the side of the base 201 corresponding to the moving beam 202 in the Y-axis direction (i.e., the first direction). In some embodiments, the moving beam driving motor 203 is securely mounted on the base 201. For example, the moving beam driving motor 203 may be securely mounted to a lower portion of the base 201 (e.g., the lower portion close to any side of the base 201 in the X-axis direction (i.e., the second direction)).

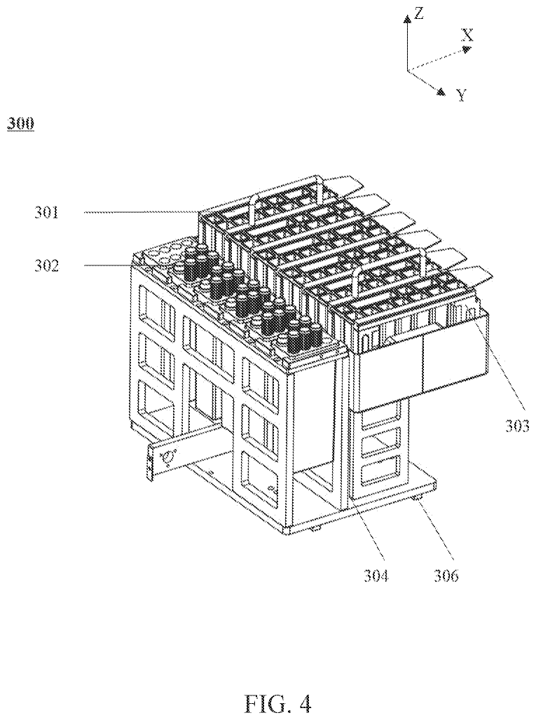

The consumable assembly 300 is configured to carry consumables used during biological sample processing. The consumables may comprise a reagent kit, a pipette tip, a target biological substance collection tube, etc. FIG. 4 is a schematic structural diagram of a consumable assembly according to some embodiments of this specification. As shown in FIG. 4, the consumable assembly 300 may comprise a reagent kit assembly 301, a pipette tip box 302 and a consumable supporting seat 304. The reagent kit assembly 301 and the pipette tip box 302 are provided on the consumable supporting seat 304. The reagent kit assembly 301 and the pipette tip box 302 are arranged in the X-axis direction (i.e., the second direction).

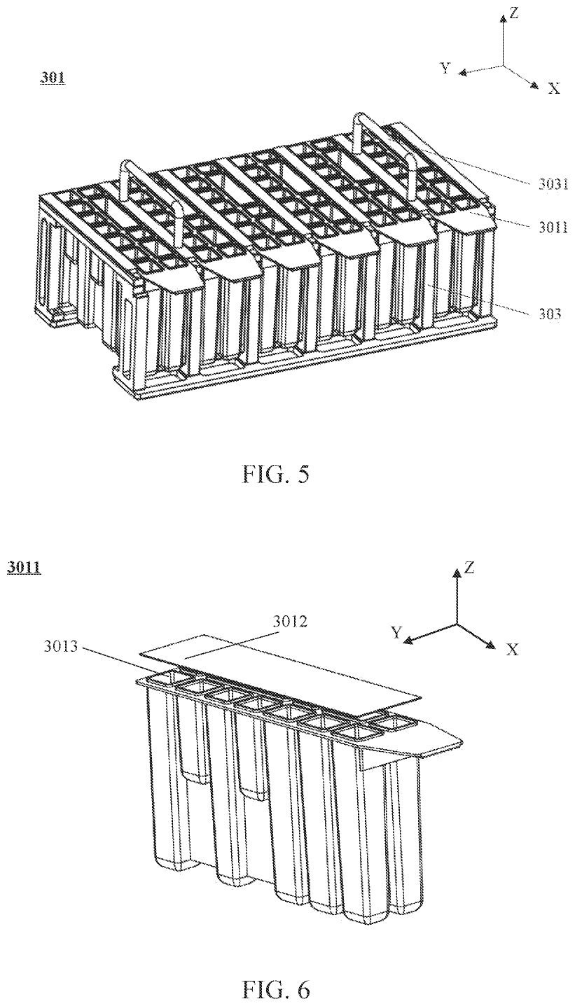

The reagent kit assembly 301 is configured to provide reagents and/or magnetic beads used during biological sample processing. FIG. 5 is a schematic structural diagram of an exemplary reagent kit assembly according to some embodiments of this specification. As shown in FIG. 5, the reagent kit assembly 301 comprises a reagent kit holder 303 and one or more reagent kits 3011. The reagent kits 3011 are placed on the consumable supporting seat 304 by means of the reagent kit holder 303. Before the biological sample processing, the one or more reagent kits can be loaded onto the reagent kit holder 303 according to the requirements of a biological sample processing process, so that during the biological sample processing, reagents and/or magnetic beads required for the processing are conveniently added to a biological sample.

The reagent kit holder 303 is configured to load the reagent kits 3011. The one or more reagent kits 3011 may be loaded on the reagent kit holder 303. For example, the one or more reagent kits 3011 may be mounted on the reagent kit holder 303 by means of snap fitting, limiting, etc. In some embodiments, one or more reagent kit holder moving members (e.g., handles 3031) are provided on the reagent kit holder 303 and configured to move the reagent kit holder 303. For example, an operator can hold the reagent kit holder 303 by means of the handles 3031. Before the biological sample processing, the operator takes out the reagent kit holder 303 from the consumable supporting seat 304 by holding the handles 3031, the operator then loads the reagent kits 3011 into the reagent kit holder 303, and later the operator mounts the reagent kit holder 303 loaded with the reagent kits 3011 to the consumable supporting seat 304. The reagent kit holder 303 facilitates integral mounting of a plurality of reagent kits 3011 to the consumable assembly 300, and also facilitates unloading of the plurality of reagent kits 3011 from the consumable assembly 300 and/or replacement of the reagent kits 3011 after the use of the automatic extraction device is completed. The operation of moving the reagent kit holder 303 is simpler by means of the reagent kit holder moving members (e.g., the handles 3031).

The reagent kit 3011 may comprise one or more cavities. When the reagent kit 3011 comprises a plurality of cavities, the plurality of cavities may be arranged in one or more rows (e.g., 1 row, 2 rows, or 3 rows) in the Y-axis direction (i.e., the first direction). Each row of cavities extends in the X-axis direction (or the second direction). By arranging the plurality of cavities in multiple rows, it is possible to avoid that the length of the reagent kit 3011 in the X-axis is excessive due to the need for too many cavities, and in turn to avoid the excessive overall length of the automatic extraction device in X-axis direction (or the second direction). FIG. 6 is a schematic structural diagram of a reagent kit according to some embodiments of this specification. As shown in FIG. 6, the reagent kit 3011 comprises a plurality of cavities 3013 arranged in two rows.

In some embodiments, the volumes of the plurality of cavities 3013 may be the same or different. For example, the cavities 3031 may be sized according to the amounts of reagents to be contained. The cavities 3013 of the reagent kit are configured to contain the reagents and/or magnetic beads required for biological sample processing. Each cavity 3013 may contain the same or a different reagent and/or magnetic bead therein. For example, a corresponding type of reagent and/or magnetic bead may be selected according to the target biological substance to be extracted and/or the technological process. The magnetic bead described here is a magnetic particle with a surface active group. Under certain conditions, the surface active group of the magnetic bead can bind to (e.g., be coupled with) a target biological substance (e.g., a substance to be extracted) or impurities (e.g., substances other than the substances to be extracted) in a biological sample. Under the action of an external magnetic field, the magnetic bead can be subjected to magnetic attraction, thereby achieving separation of the active substance from the impurities and/or purification of the active substance. In some embodiments, the corresponding type of magnetic bead may be selected according to the particle size, the dispersity, the magnetic response time, and the coating matrices of the magnetic bead. The type of the magnetic bead is not limited here. The types of reagents may include solvents for reacting with biological samples and for washing magnetic bead conjugates, reagents for eluting magnetic bead conjugates to separate magnetic beads, etc. The types of reagents are not limited here. Taking plasmid extraction by means of bacterial sludge as an example, the reagent may include a bacterial sludge lysis solution, a washing solution, an eluent, etc. The magnetic bead may include a magnetic bead capable of binding to impurities obtained after bacterial sludge is lysed, a magnetic bead capable of binding to plasmids, etc.

In some embodiments, the reagent kit 3011 further comprises a sealing layer. The sealing layer can be used to seal the cavities 3013 so as to prevent the reagents in the cavities from being spilled and/or deteriorating (e.g., reacting with gases in the air) when the reagent kit 3011 is pipetted. In some embodiments, one reagent kit 3011 may correspond to one or more sealing layers. For example, the sealing layer may be a sealing layer 3012 as shown in FIG. 6, and one sealing layer 3012 can be used to seal a plurality of cavities 3013 of one reagent kit 3011. For another example, the sealing layers may correspond to the cavities of the reagent kit 3011 on a one-to-one basis, and the plurality of cavities of the reagent kit 3011 may be respectively sealed with separate sealing layers. In some embodiments, the sealing layer may be in the form of a sealing film. The sealing film may be an aluminum film, a plastic film, etc., and the material of the sealing film is not limited here.

The pipette tip box 302 is configured to provide a pipette tip and/or a target biological substance collection tube required for pipetting during the biological sample processing. In some embodiments, the pipette tip box 302 corresponds to the reagent kit 3011 on a one-to-one basis. That is, one reagent kit 3011 corresponds to one pipette tip box 302. By means of the one-to-one correspondence between the pipette tip box 302 and the reagent kit 3011, when a plurality of reagent kits 3011 are provided, a pipetting operation can be performed on the plurality of reagent kits 3011 simultaneously, thereby simplifying the operation of the automatic extraction device and shortening the time required by the automatic extraction process. FIG. 7 is a schematic structural diagram of a pipette tip box according to some embodiments of this specification. As shown in FIG. 7, the pipette tip box assembly comprises one or more pipette tips 3021, a box body 3022 having an upper end open, a cover plate 3023, and one or more target biological substance collection tubes 3026. The cover plate 3023 is connected to an opening of the box body 3022. The pipette tips 3021 and the target biological substance collection tubes 3026 are loaded on the cover plate 3023 and the box body 3022 by means of through holes 3025 provided in the cover plate. In some embodiments, the through hole 3025 may be sized to allow the pipette tips 3021 to be placed vertically or approximately vertically, so as to facilitate the fitting between tail portions of the pipette tips 3021 and the pipetting apparatus 400. Before the biological sample processing, the one or more pipette tips 3021 and target biological substance collection tubes 3026 can be loaded onto the cover plate 3023 and the box body 3022 according to the requirements of a biological sample processing, so that during the biological sample processing, reagents, magnetic beads and/or waste liquids required for the processing are conveniently added to/removed from the biological sample.

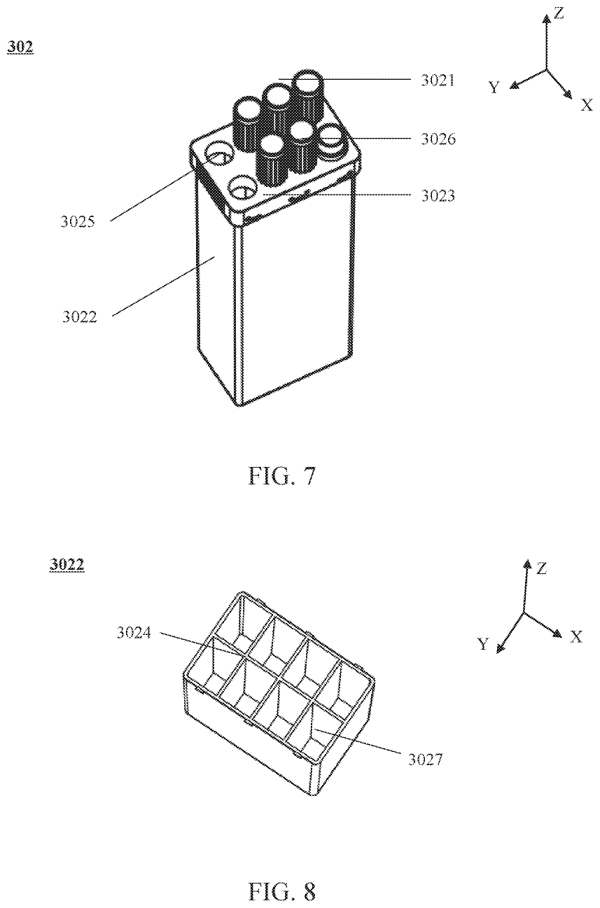

The box body 3022 and the cover plate 3023 are configured to load the one or more pipette tips 3021 and target biological substance collection tubes 3026. The inside of the box body 3022 is divided into a plurality of accommodating cavities by partitions, and the cover plate 3023 is provided with a plurality of through holes 3025 corresponding to the plurality of accommodating cavities. For example, a single through hole 3025 corresponds to an opening of a single accommodating cavity. At least some of the accommodating cavities and the through holes 3025 corresponding thereto are configured to load the one or more pipette tips 3021. In some embodiments, the plurality of accommodating cavities of the box body 3022 may be arranged in one or more rows (e.g., 1 column, 2 columns, or 3 columns) in the Y-axis direction (i.e., the first direction). Each row of accommodating cavities extends in the X-axis direction (i.e., the second direction). FIG. 8 is a schematic diagram of an internal structure of a pipette tip box according to some embodiments of this specification. As shown in FIGS. 7 and 8, the box body 3022 comprises a plurality of partitions 3024 that divide the box body 3022 into a plurality of accommodating cavities 3027. The plurality of accommodating cavities 3027 are arranged in two rows in the Y-axis direction (i.e., the first direction). By loading one pipette tip 3021 in one accommodating cavity 3027, it is possible to prevent contaminants such as aerosol generated in the box body 3022 by a pipette tip that have pipetted different reagents from affecting the biological sample processing process.

The pipette tip 3021 is used in combination with the pipetting apparatus 400 to aspirate different reagents, magnetic beads and/or waste liquids during the biological sample processing. The plurality of pipette tips 3021 may be arranged in one or more rows in the Y-axis direction (i.e., the first direction). Each row of pipette tips extends in the Y-axis direction (i.e., the first direction). In some embodiments, the number of rows of the plurality of pipette tips 3021 may be the same as or different from the number of rows of the cavities 3013 of the reagent kit 3011 corresponding thereto. For example, the plurality of pipette tips 3021 are arranged in two rows, and the cavities 3013 of the reagent kit 3011 corresponding thereto are arranged in three rows. For another example, the plurality of pipette tips 3021 and the cavities 3013 of the reagent kit 3011 corresponding thereto are both arranged in three rows. In some embodiments, the pipette tips 3021 are loaded in the corresponding accommodating cavities 3027 by means of the through holes 3025 of the cover plate 3023. As shown in FIGS. 7 and 8, tip portions of the pipette tips 3021 for liquid aspiration pass through the through holes 3025 and are placed in the accommodating cavities 3027 corresponding to the through holes 3025, and tail portions of the pipette tips 3021 fitting with the pipetting apparatus 400 are placed outside the accommodating cavities 3027. In some embodiments, the plurality of pipette tips 3021 may be the same or may not completely the same. For example, the pipette tip 3021 may be sized according to the amount of liquid to be pipetted by the pipette tip 3021. For another example, the pipette tips 3021 with different structures may be provided to adapt to different reagents, facilitating the smooth liquid pipetting of each reagent. In some embodiments, a single pipette tip 3021 may be used once or repeatedly during the biological sample processing. For example, at two stages during the biological sample processing where the same reagent needs to be pipetted from the reagent kit 3011, the same pipette tip 3021 may be repeatedly used to pipette the same reagent. That is, the pipette tip 3021 may not be provided in a one-to-one correspondence with the cavities 3013 of the reagent kit 3011, and the cavities 3013 containing the same reagent may correspond to the same pipette tip 3021. For another example, in order to make the amount of pipetted reagent more accurate or avoid mutual contamination, each pipette tip 3021 is only used to pipette the liquid once. That is, the pipette tips 3021 may be provided in one-to-one correspondence with the cavities 3013 of the reagent kit 3011, so as to pipette reagents from different cavities 3013 with different pipette tips 3021.

The target biological substance collection tube 3026 is configured to contain a target biological substance obtained by means of extraction, separation and/or purification during the biological sample processing. In some embodiments, the pipette tip box 302 may comprise one or more target biological substance collection tubes 3026. For example, the number and/or the size of target biological substance collection tubes may be determined according to the amount of target biological substances that can be collected. In some embodiments, the target biological substance collection tubes 3026 are loaded in the corresponding accommodating cavities 3027 by means of the through holes 3025 of the cover plate 3023. As shown in FIG. 7, at least part of the target biological substance collection tube 3026 is located above the corresponding through hole 3025, to facilitate the pipetting of the target biological substance into the target biological substance collection tube 3026.

In some embodiments, the consumable assembly 300 may further comprise a consumable assembly driving motor. The consumable assembly driving motor 305 is configured to drive the consumable assembly 300 to move in the Y-axis direction (i.e., the first direction), so as to adjust a position of the consumable assembly 300 and facilitate a pipetting operation. For example, when the number of rows of the reagent kits 3011 is inconsistent with the number of rows of the pipette tip boxes 302, pipetting can be achieved by moving the entire consumable assembly 300 multiple times. For example, the consumable assembly 300 is first moved a position where the pipetting apparatus 400 can reach a pipette tip 3021, so as to assemble the pipette tip 3021, and the consumable assembly 300 and the pipetting apparatus 400 are then moved such that the assembled pipette tip 3021 can pipette a reagent from the reagent kit 3011. In some embodiments, the consumable assembly driving motor 305 is in transmission connection with the consumable supporting seat 304. In some embodiments, the consumable assembly driving motor 305 may be a lead screw motor. For example, the consumable assembly driving motor 305 may be in transmission connection with the consumable assembly driving motor 305 by means of a consumable assembly lead screw-nut pair (similar to the moving beam lead screw-nut pair), and the consumable assembly driving motor 305 drives the consumable assembly lead screw-nut pair to rotate, so as to drive the consumable assembly 300 to move in the Y-axis direction (i.e., the first direction). In some embodiments, the consumable assembly driving motor 305 may be mounted and secured on the base 201. In some embodiments, the consumable assembly 300 may comprise a plurality of consumable assembly driving motors. For example, the consumable assembly driving motor 305 may comprise a first consumable assembly driving motor and a second consumable assembly driving motor. The first consumable assembly driving motor is configured to drive the reagent kit assembly 301 to move in the Y-axis (i.e., the first direction). The second consumable assembly driving motor is configured to drive the pipette tip box 302 to move in the Y-axis (i.e., the first direction). By means of the plurality of consumable assembly driving motors, when the number of rows of the reagent kits 3011 is inconsistent with the number of rows of pipette tip boxes 302, the pipette tip boxes 302 and/or the reagent kit assembly 301 can be moved separately to make the pipetting operation more efficient. For example, when the number of rows of the reagent kits 3011 is inconsistent with the number of rows of pipette tip boxes 302, the pipetting apparatus 400 and the pipette tip box 302 may be moved first to assemble a pipette tip 3021, and the reagent kit assembly 301 and the pipetting apparatus 400 may then be moved to enable the assembled pipette tip 3021 to pipette a reagent from the reagent kit 3011. For another example, when the number of rows of the reagent kits 3011 is inconsistent with the number of rows of pipette tip boxes 302, the pipette tip box 302 and the reagent kit assembly 301 may be moved simultaneously, such that after the pipetting apparatus 400 is assembled with a pipette tip 3021, the pipetting apparatus can be moved along the X-axis and then pipette a reagent from the reagent kit 3011.

In some embodiments, the consumable supporting seat 304 may be connected to the base 201 by means of a consumable assembly guide rail 306. In some embodiments, the consumable assembly guide rail 306 may be a linear guide rail. The consumable assembly guide rail extends in the Y-axis direction (i.e., the first direction). The consumable assembly 300 may be moved on the consumable assembly guide rail under the drive of the consumable assembly driving motor. In some embodiments, the consumable assembly guide rail (e.g., second linear guide rail 306) is located at the lower portion of the consumable supporting seat 304. In some embodiments, the consumable assembly guide rail (e.g., the second linear guide rails 306) may be mounted and secured on the base 201. In some embodiments, the consumable assembly 300 may comprise a plurality of consumable assembly guide rails (e.g., two second linear guide rails 306 as shown in FIG. 4). By means of the plurality of consumable assembly guide rails, the consumable assembly 300 may be stably moved without deviating from a preset direction. In some embodiments, when the consumable assembly 300 comprises a plurality of consumable assembly driving motors, each consumable assembly driving motor may respectively correspond to one or more respective consumable assembly guide rails.

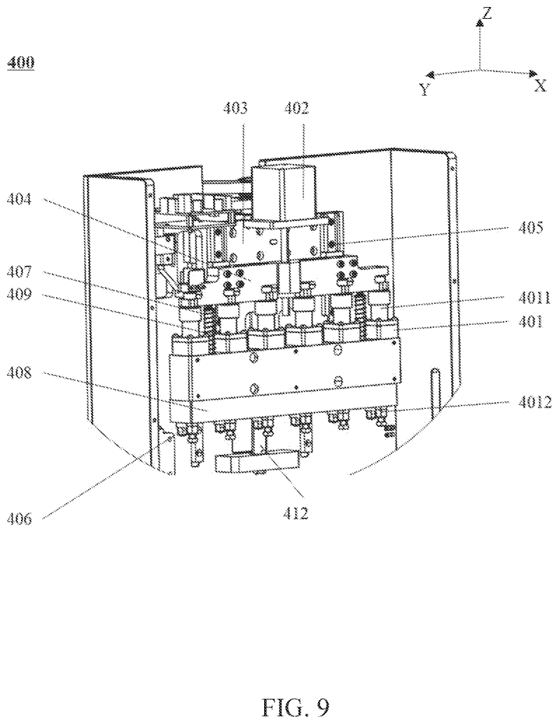

The pipetting apparatus 400 is configured to implement the pipetting operation during the biological sample processing. For example, the pipetting apparatus 400 may be configured for the pipetting operation between the consumable assembly 300, the sample processing apparatus 100, and/or the waste liquid apparatus 500. FIG. 9 is a schematic structural diagram of a pipetting apparatus according to some embodiments of this specification. As shown in FIG. 9, the pipetting apparatus 400 comprises one or more pipettes 401. The one or more pipettes 401 are arranged in the Y-axis direction (i.e., the first direction).

The pipette 401 can be detachably and sealingly connected to the pipette tip 3021 in the pipette tip box 302, thereby implementing replacement of the pipette tip 3021 for the pipette 401. When the pipette 401 is sealingly connected to the pipette tip 3021, the pipette 401 can achieve liquid aspiration and dispensing. After the pipette 401 completes the liquid dispensing, the pipette tip 3021 sealingly connected thereto may be detached and placed in the accommodating cavity 3027 corresponding to the pipette tip. In some embodiments, the structure of the pipette 401 may be a pipette tip structure, a syringe structure, or other structures that can perform liquid aspiration and dispensing operations. In some embodiments, the pipette 401 comprises a cavity and a piston 4011. The cavity may be detachably connected to the pipette tip 3021. For example, the cavity has a suction head 4012, and the pipette 401 implements detachable sealed connection with the pipette tip 3021 by means of the suction head 4012. In some embodiments, the suction head 4012 is aligned to a row of pipette tips 3021 extending in the X-axis direction (i.e., the second direction), such that the suction head 4012 is detachably and sealingly connected to the pipette tips 3021. A first end (e.g., a lower end) of the piston 4011 may seal the cavity. The piston 4011 can move up and down relative to the cavity (e.g., move up and down in a direction parallel to the Z-axis (i.e., the third direction)). When the piston 4011 moves up in the Z-axis direction (i.e., the third direction), the pipette 401 can control the pipette tip 3021 to aspirate a liquid. When the piston moves down in the Z-axis direction (the third direction), the pipette 401 can control the pipette tip 3021 to dispense the liquid.

In some embodiments, the pipetting apparatus 400 further comprises a piston driving assembly. For example, the piston driving assembly comprises a piston driving motor 402, a driving plate 403, a piston guide rail 405, etc. or any combination thereof. The piston driving motor 402 is configured to drive the piston 4011 to move up and down in the direction parallel to the Z-axis (i.e., the third direction). In some embodiments, the piston driving motor 402 may be a lead screw motor. The driving plate 403 is configured to be securely connected to a second end (e.g., an upper end) of the piston 4011. By means of the transmission connection between the driving plate 403 and the piston driving motor 402, the piston driving motor 402 can drive the driving plate 403 to move in the Z-axis direction (i.e., the third direction), so as to drive the piston 4011 to move in the Z-axis direction (i.e., the third direction). In some embodiments, the pipetting apparatus 400 may further comprise a support plate 404. The support plate 404 is configured to mount and secure one or more assemblies (e.g., the cavity, the piston driving motor 402, or the piston guide rail 405) of the pipetting apparatus 400. For example, the piston guide rail 405 may be disposed on the support plate 404 in the Z-axis direction (i.e., the third direction), the driving plate 403 is connected to the piston guide rail 405, and the driving plate 403 can move on the piston guide rail 405 under the drive of the piston driving motor 402, so as to drive the piston 4011 to move up and down so as to implement liquid aspiration and dispensing. In some embodiments, the piston guide rail 405 may include a linear guide rail.

In some embodiments, the pipetting apparatus 400 further comprises a piercing mechanism 406. The piercing mechanism 406 is configured to pierce the sealing layer (for example, the sealing layer 3012) of the reagent kit 3011, such that the pipette tip 3021 sealingly connected to the pipette 401 can pass through the sealing layer to aspirate the reagent from the reagent kit 3011. The piercing mechanism 406 correspond to the pipette 401 on a one-to-one basis. For example, one piercing mechanism 406 corresponds to one pipette 401. In some embodiments, the piercing mechanism 406 comprises a sharp head for piercing the sealing layer. For example, the piercing mechanism 406 may be of a magnetic film knife structure. In some embodiments, the pipetting apparatus 400 may further comprise a push plate 408 and a push rod 409. The push rod 409 is connected to the push plate 408, and a spring 407 is sleeved on the push rod 409. Before the pipette 401 pipettes the reagent from the reagent kit 301, when the driving plate 403 pushes the push rod 409 to move down in the direction parallel to the Z-axis (i.e., the third direction), the push rod 409 pushes the push plate 408 to move down and compresses the spring 407, and when the driving plate 403 moves away from the push rod, the compressed spring 407 provides a restoring elastic force such that the push plate 408 is restored by means of the restoring force of the spring 407. In some embodiments, the push plate 408 is located above the suction head 4012 and the piercing mechanism 406. The piercing mechanism 406 and the suction head 4021 are disposed in the X-axis direction (i.e., the second direction) or the Y-axis direction (i.e., the first direction). When the driving plate 403 pushes the push rod 409 to move down, the push rod 409 pushes the push plate 408 to move down, and the suction head 4021 of the pipette 401 may come into contact with and be sealingly connected to the pipette tip 3021, or the piercing mechanism 406 pierces the sealing layer of the reagent kit 3011.

In some embodiments, the pipetting apparatus 400 may further comprise a pipetting apparatus driving assembly. The pipetting apparatus driving assembly comprises a pipetting apparatus driving motor 410, a pipetting apparatus guide rail 411, etc. or any combination thereof. The pipetting apparatus driving motor may be in transmission connection with the support plate 404. Under the drive of the pipetting apparatus driving motor 410, the support plate 404 can move up and down relative to the moving beam 202 in the Z-axis direction (i.e., the third direction), such that the entire pipette 401 can move up and down in the Z-axis direction (i.e., the third direction). In some embodiments, the pipetting apparatus driving motor 410 may be a lead screw motor. In this case, the pipetting apparatus 400 further comprises a pipette lead screw-nut pair 412 and a second synchronous pulley assembly 413. The pipette lead screw-nut pair 412 may be connected to the pipetting apparatus 400 (e.g., the support plate 404). The pipette lead screw-nut pair 412 may be connected to the pipetting apparatus driving motor 410 via the second synchronous pulley assembly 413, and the pipetting apparatus driving motor 410 drives the pipette lead screw-nut pair 412 to rotate so as to drive the support plate 404 to move in the Z-axis direction (i.e., the third direction), such that the pipette 401 can move up and down in the Z-axis direction (i.e., the third direction). In some embodiments, the pipetting apparatus 400 further comprises the pipetting apparatus guide rail 411. The pipetting apparatus guide rail 411 may be a linear guide rail. The pipetting apparatus guide rail 411 may be mounted and secured on the moving beam 202 in the Z-axis direction (i.e., the third direction). The support plate 404 can be connected to the moving beam 202 via the pipetting apparatus guide rail 411. Under the drive of the pipetting apparatus driving motor 410, the support plate 404 can move up and down along the pipetting apparatus guide rail 411, thereby driving the pipette 401 to move up and down in the Z-axis direction (i.e., the third direction).

The waste liquid apparatus 500 is configured to contain a waste liquid generated during the biological sample processing. The waste liquid apparatus 500 may comprise a waste liquid box 501 and a waste liquid box holder 502. The waste liquid box 501 is detachably mounted on the waste liquid box holder 502. The waste liquid box 501 is fixed relative to the base 201. For example, the waste liquid box 501 may be mounted on the sample processing apparatus 100 by means of the waste liquid box holder 502. In some embodiments, the waste liquid box 501 may comprise one waste liquid box body and one waste liquid box cover plate. The waste liquid box body is open at the upper end. One or more through holes 503 are provided in the waste liquid box cover plate to facilitate transferring of the waste liquid into the waste liquid box 501. The one or more through holes 503 correspond to sample tubes 110 and/or reaction tubes 120 in the sample processing apparatus 100 on a one-to-one basis. For example, if the sample processing apparatus 100 comprises six sample tubes, the waste liquid box cover plate comprises six through holes. In some embodiments, the waste liquid box 501 may comprise a plurality of waste liquid box bodies and a plurality of corresponding waste liquid box cover plates. The plurality of waste liquid box bodies correspond to a plurality of sample tubes 110 and/or the reaction tubes 120 in the sample processing apparatus 100 on a one-to-one basis. The plurality of waste liquid box cover plates are each provided with a through hole. For example, if the sample processing apparatus 100 comprises six sample tubes, the waste liquid box 501 comprises six waste liquid box bodies corresponding to the six sample tubes respectively. In some embodiments, the waste liquid box 501 may not comprise the waste liquid box cover plate.

The sample processing apparatus 100 is configured to perform uniform mixing processing and magnetic attraction processing on a biological sample. In some embodiments, the sample processing apparatus 100 may further comprise a uniform mixing mechanism and a magnetic attraction mechanism. The uniform mixing mechanism is configured to perform uniform mixing processing on the biological sample during processing. The magnetic attraction mechanism is configured to perform magnetic attraction processing on the biological sample during processing. In some embodiments, the sample processing apparatus 100 may further comprise a sample tube and a reaction tube. The sample tube may be configured to contain the biological sample, and the reaction tube may be configured to contain a solution obtained after processing in the sample tube. In some embodiments, the volume of the sample tube may be equal to or greater than the volume of the reaction tube. In some embodiments, the sample tube may have the same height as the reaction tube. In some embodiments, the sample tube and/or the reaction tube may be in the shape of a test tube or be of a box structure with an open top, which is not limited here.

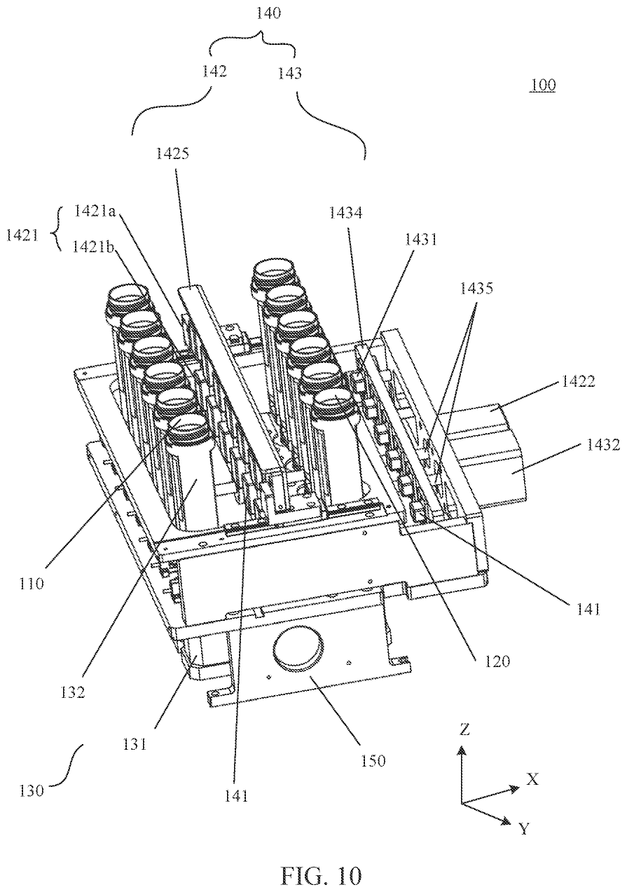

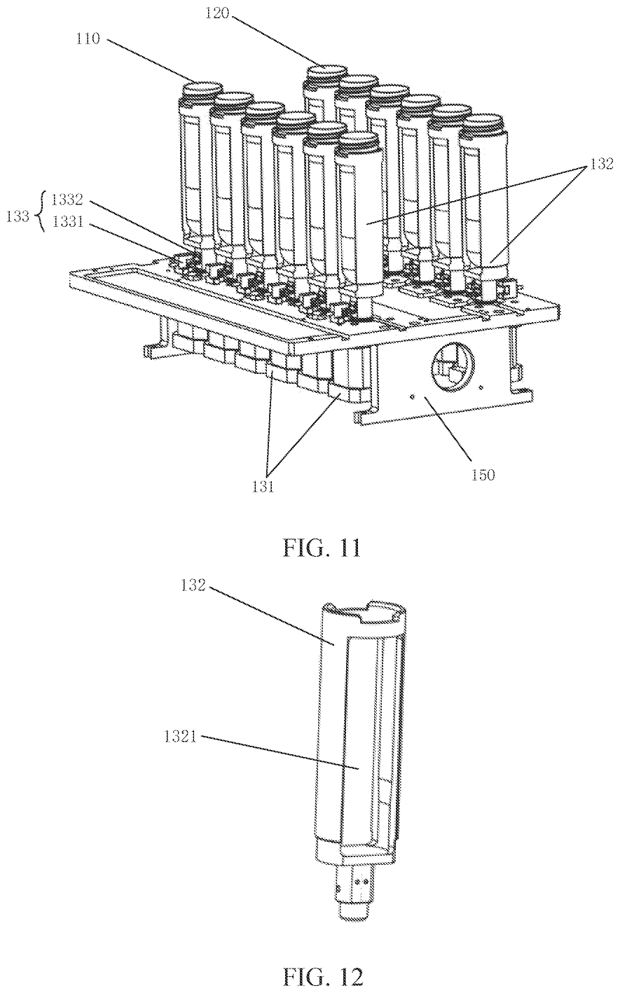

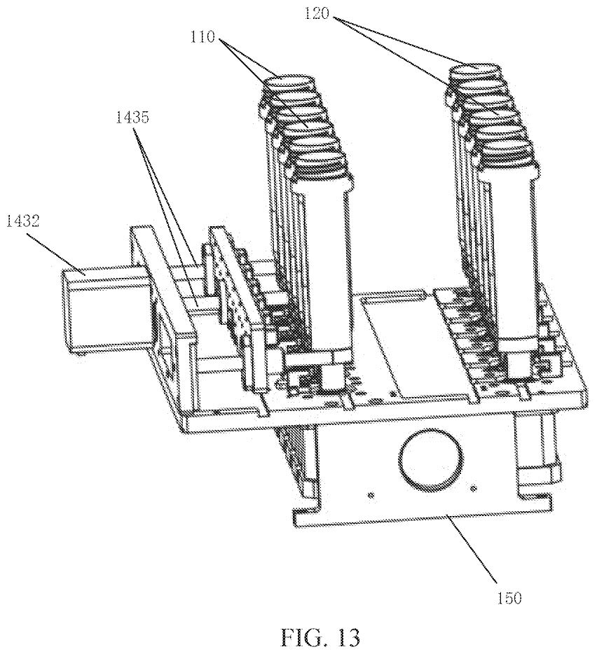

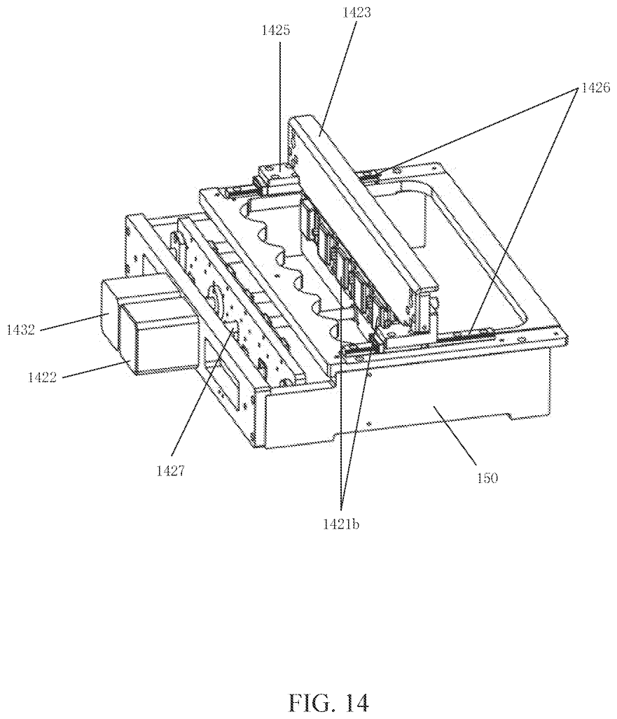

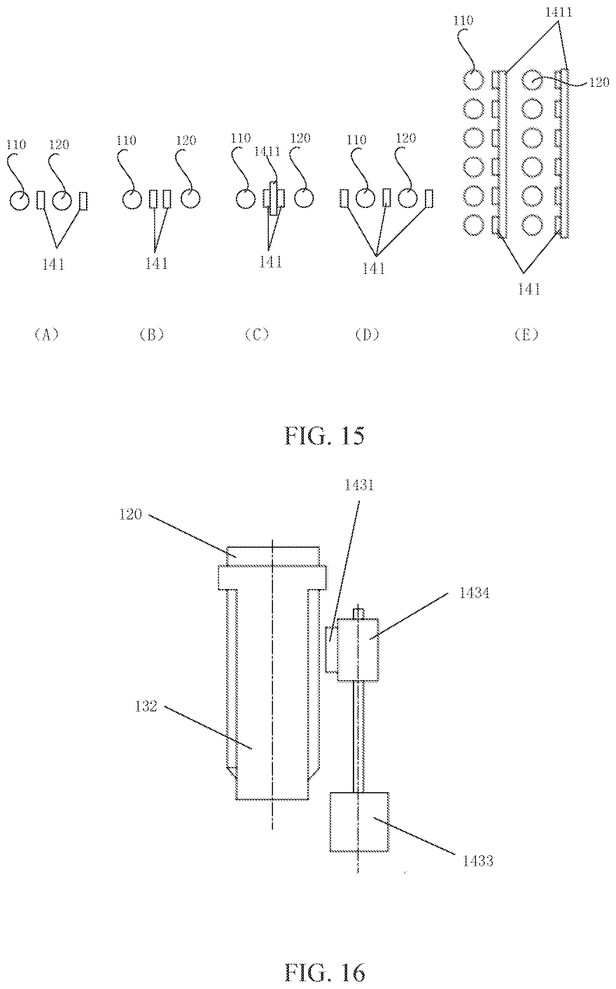

FIG. 10 is a schematic structural diagram of a sample processing apparatus according to some embodiments of this specification. FIG. 10 is a schematic structural diagram of a uniform mixing mechanism according to some embodiments of this specification. FIG. 12 is a schematic structural diagram of a tube holder according to some embodiments of this specification. FIG. 13 is a schematic structural diagram of a magnetic attraction mechanism according to some embodiments of this specification. FIG. 14 is a schematic structural diagram of a magnetic attraction mechanism according to some other embodiments of this specification. FIG. 15 is a schematic structural diagram of the arrangement of magnets of a magnetic attraction mechanism according to some embodiments of this specification. FIG. 16 is a demonstration diagram of magnetic attraction of a magnetic attraction mechanism according to some embodiments of this specification. FIG. 17 is a demonstration diagram of magnetic attraction of a magnetic attraction mechanism according to some embodiments of this specification.

The sample processing apparatus 100 may comprise a uniform mixing mechanism 130 and a magnetic attraction mechanism 140. The sample processing apparatus 100 may further comprise at least one of a sample tube 110 and a reaction tube 120.

The sample tube 110 is a container configured to contain a biological sample, perform biological sample preprocessing, and/or perform biological sample reaction. The reaction tube 120 is a container configured to contain a biological sample, perform biological sample preprocessing, and/or perform biological sample reaction. In some embodiments, the sample processing apparatus 100 may only comprise the sample tube 110, that is, only the sample tube 110 is provided for sample processing. In some embodiments, the sample processing apparatus 100 may comprise only the reaction tube 120, that is, only the reaction tube 120 is provided for sample processing. In some embodiments, the sample processing apparatus 100 may comprise a sample tube 110 and a reaction tube 120 that are jointly used to perform sample processing work. In some embodiments, the biological sample preprocessing may comprise biological culture, resuspension, magnetic attraction, separation, or any other feasible biological preprocessing operations. In some embodiments, the biological sample reaction may comprise resuspension, magnetic attraction, separation, lysis, precipitation or any other feasible biological sample reaction operations. In some embodiments, the sample tube 110 may be configured to contain a biological sample to be reacted. For example, the sample tube 110 may be configured to contain a biological sample to be placed into the reaction tube 120 for reaction. In some embodiments, the reaction tube 120 may be configured to contain a biological sample transferred from the sample tube 110. For example, the reaction tube 120 may be configured to contain the biological sample that is transferred from the sample tube 110 and has been subjected to the biological sample preprocessing.

In some embodiments, the sample tube 110 and the reaction tube 120 may have the same structure and/or volume. For example, tube bodies of the sample tube 110 and the reaction tube 120 are both in the shape of a straight tube. For another example, the volumes of the sample tube 110 and the reaction tube 120 are both 100 ml. In some embodiments, the sample tube 110 and the reaction tube 120 may have different structures and/or volumes. In a specific embodiment, specification parameters of the sample tube 110 or the reaction tube 120, such as structural form and volume may be set in any feasible manners, which are not particularly limited in the embodiments of this specification.

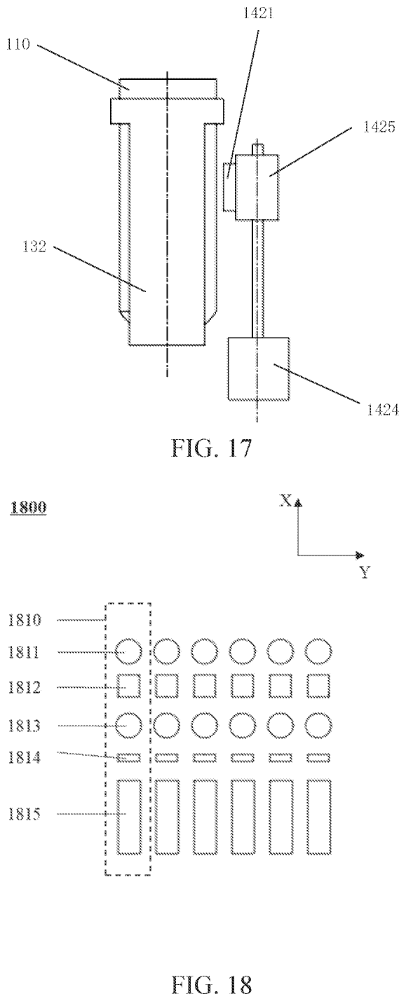

In some embodiments, the sample processing apparatus 100 may comprise one sample tube 110 and one reaction tube 120. In some embodiments, the sample processing apparatus 100 may comprise one sample tube 110 and a plurality of reaction tubes 120. In some embodiments, the sample processing apparatus 100 may comprise a plurality of sample tubes 110 and a plurality of reaction tubes 120, so as to implement processing of a plurality of samples and improve the processing efficiency of the biological samples. In some embodiments, the number of sample tubes 110 or reaction tubes 120 may be 6, 8 or any other numbers. In some embodiments, the sample processing apparatus 100 may be provided with a plurality of processing channels arranged at intervals, and each processing channel may comprise one sample tube 110 and at least one reaction tube 120, that is, one or more (at least two) reaction tubes 120 may be provided. Each processing channel here may be understood as a unit sample processing element in a sample processing system, because a sample processing process of a certain category or a certain time can be implemented by typically providing one sample tube 110 and at least one reaction tube 120 in each unit sample processing element. A further description of the processing channels may be seen with reference to FIG. 18 and an illustration thereof.

When a plurality of sample tubes 110 and a plurality of reaction tubes 120 are provided, a separate channel configuration having different processing channels not only expands the scale of sample processing, but also facilitates the separate management of each sample processing channel. For example, each processing channel may be configured differentially. In some embodiments, differential configurations of the processing channels may be determined according to the characteristics of the sample processing process. For example, the characteristics of the sample processing process may comprise a required number of reaction tubes 120, a required volume of the sample tube 110 or the reaction tube, etc., and may be determined according to a sample throughput (e.g., a larger sample throughput exceeding a certain threshold, or a smaller sample throughput less than a certain threshold), a sample processing type (e.g., a plasmid purification process or a protein extraction process), or any other feasible sample processing parameters.

In some embodiments, the processing channels may be arranged parallel to each other. In some embodiments, the processing channels may be disposed in a longitudinal or transverse arrangement. In some embodiments, the sample tubes 110 and the reaction tubes 120 of each processing channel may be sequentially provided in parallel in the (illustrated) X-axis direction (i.e., the second direction). Illustratively, as shown in FIG. 9, the sample processing apparatus 100 comprises six channels, the sample tubes 110 and the reaction tubes 120 in each channel are disposed at intervals in parallel in the X-axis direction (i.e., the second direction), and the plurality of channels are arranged in parallel in the Y-axis direction (i.e., the first direction). In some embodiments, a predetermined interval is provided between the sample tube 110 and the reaction tube 120.

The uniform mixing mechanism 130 is configured to perform uniform mixing processing on the biological sample in the sample tube 110 and/or the biological sample in the reaction tube 120, so as to achieve a uniform mixing effect on the biological sample. In some embodiments, the uniform mixing mechanism 130 may be only configured to perform uniform mixing processing on the biological sample in the sample tube 110. In some embodiments, the uniform mixing mechanism 130 may be only configured to perform uniform mixing processing on the biological sample in the reaction tube 120. In some embodiments, the uniform mixing mechanism 130 may be configured to simultaneously or sequentially perform uniform mixing processing on the biological sample in the sample tube 110 and the biological sample in the reaction tube 120.

In some embodiments, the uniform mixing mechanism 130 may adopt an electric motor driving mode (e.g., electrical transmission driving or shaking table electromagnetic driving) or any other feasible driving modes (such as hydraulic driving, or pneumatic driving). In some embodiments, the uniform mixing mechanism 130 may comprise a uniform mixing driving motor 131 and a tube holder 132. The uniform mixing driving motor 131 is in transmission connection with the tube holder 132, the sample tube 110 and/or the reaction tube 120 are provided in the tube holder 132, and the uniform mixing driving motor 131 can drive the tube holder 132 to move, so as to driving the biological sample in the sample tube 110 and/or the biological sample in the reaction tube 120 to undergo uniform mixing processing.

The tube holder 132 is configured to place the sample tube 110 and/or the reaction tube 120 and can drive the sample tube 110 and/or the reaction tube 120 to move. In some embodiments, the tube holder 132 may be of a cylindrical structure sleeved outside the sample tube 110 or the reaction tube 120. In some embodiments, the sample tube 110 or the reaction tube 120 may be freely sleeved on the tube holder 132 or freely removed from the tube holder 132. In some embodiments, the sample tube 110 or the reaction tube 120 may be mounted to the tube holder 132 in a snap-on connection manner. In some embodiments, the sample tube 110 or the reaction tube 120 is detachably connected to the tube holder 132.

In some embodiments, the tube holder 132 is connected to a rotating shaft (not shown) of the uniform mixing driving motor 131, and under the action of the rotating shaft, the uniform mixing driving motor 131 can drive the tube holder 132 to rotate axially such that the tube holder 132 drives the sample tube 110 and/or the reaction tube 120 to rotate axially, achieving the uniform mixing processing of the biological sample. Such an axial rotation-based driving mode can implement sufficient uniform mixing of the biological sample in the sample tube 110 and/or the reaction tube 120, and improve the efficiency of uniform mixing processing. In some embodiments, a driving rotation mode for the uniform mixing driving motor 131 and the tube holder 132 may also be set to any other feasible structural modes. For example, the uniform mixing driving motor 131 may drive the tube holder 132 to rotate at multiple angles (e.g., 360 degrees) around a central line of the tube holder 132, and may also drive the tube holder 132 to rotate at a constant or variable tilt angle within a corresponding plane (e.g., a horizontal plane). In some embodiments, the uniform mixing driving motor 131 can drive the tube holder 132 to rotate in an axial direction for a period of time and then rotate in an opposite axial direction (e.g., first axially rotate clockwise, and then axially rotate counterclockwise), such that the tube holder 132 drives the sample tube 110 and/or the reaction tube 120 to axially rotate in changing directions. Such an arrangement of axial rotation in changing directions can ensure that the biological sample in the sample tube 110 and/or the reaction tube 120 are fully mixed uniformly, thereby improving the efficiency of uniform mixing processing. In some embodiments, the rotation speed of the uniform mixing driving motor 131 may be set differently according to specific circumstances, so as to driving the tube holder 132 to rotate at different speeds (e.g., at a high speed, or at a low speed). For example, the rotation speed may be set to 1500 r/min.

In some embodiments, the uniform mixing mechanism 130 may comprise a plurality of uniform mixing driving motors 131 and a plurality of tube holders 132. The plurality of uniform mixing driving motors 131 are in transmission connection with the plurality of tube holders 132 in one-to-one correspondence, and one sample tube 110 or one reaction tube 120 is provided in each tube holder 132. With such an arrangement, it is possible to implement driving of the sample tubes or the reaction tubes in the plurality of tube holders in various changing modes, and different driving modes of different uniform mixing driving motors 131 may be respectively set for the corresponding tube holders 132. The driving modes here may be determined according to driving parameters such as a driving force parameter, a driving start time, and start and stop times. It is also possible to set specific driving modes for some uniform mixing driving motors 131 of the plurality of uniform mixing driving motors 131, especially in a sample processing scenario that needs a plurality of processing channels, to better implement targeted settings adapted to various uniform mixing processing situations, thereby making the uniform mixing processing more efficient during sample processing and meeting diverse sample processing demands. In some embodiments, each uniform mixing driving motor 131 can be controlled separately to drive the corresponding tube holder 132 to move, such that respective separate control of the uniform mixing driving motors is facilitated, and personalized uniform mixing demands are met.

The magnetic attraction mechanism 140 is configured to perform magnetic attraction on the biological sample in the sample tube 110 and/or the biological sample in the reaction tube 120 so as to facilitate a biological separation operation. The magnetic attraction mechanism 140 may comprise a magnet 141 that can move relative to the sample tube 110 and/or the reaction tube 120 so as to perform magnetic attraction processing on the biological sample in the sample tube 110 and/or the biological sample in the reaction tube 120. In some embodiments, the magnet 141 has a predetermined magnetic attraction action area. In some embodiments, the magnetic attraction action area of the magnet 141 may be the area of a magnetic attraction action surface facing the sample tube 110 and/or the reaction tube 120 for magnetic attraction processing.

In some embodiments, in a single processing channel, one magnet 141 may be provided on the other side of the sample tube 110 away from the reaction tube 120 in the X-axis direction. In some embodiments, between the processing channels, one magnet 141 may be provided between two sample tubes 110 in the Y-axis direction. In some embodiments, for the plurality of processing channels, one magnet 141 may be provided on the outermost side of any one of the sample tubes 110 at two ends in the Y-axis direction. In some embodiments, in a single processing channel, one magnet 141 or two corresponding magnets 141 may be provided between the sample tube 110 and the reaction tube 120 that are adjacent to each other, or between two adjacent reaction tubes 120. In some embodiments, the two corresponding magnets 141 may be interspersed between the sample tube 110 and the reaction tube 120 that are adjacent to each other, or between two adjacent reaction tubes 120. In some embodiments, the magnet 141 may be provided on a magnetic attraction bracket 1411 of the magnetic attraction mechanism 140, and the magnetic attraction bracket 1411 may be of any feasible bracket structure as long as it can carry the magnet 141. Only by way of example, FIG. 14 illustrates several layouts (A), (B), (C), (D) and (E) of a magnetic attraction mechanism 140. Specifically, it is possible that as demonstrated in (A), two magnets 141 are each disposed on the same side of the two tubes (the sample tube 110 and the reaction tube 120) and are spaced apart from each other, it is also possible that as demonstrated in (B), two magnets 141 are provided adjacent to each other between the two tubes, it is also possible that as demonstrated in (C), the two magnets 141 in (B) are provided on opposite sides of the magnetic attraction bracket 1411, it is also possible that a plurality of magnets 141 are provided between two tubes and on respective outer sides of the two tubes, or it is also possible that magnets are provided in a multi-channel array arrangement as shown in (E). It should be noted that the magnetic attraction mechanism 140 may also use any other feasible forms, as long as they can implement the magnetic attraction processing of the sample tube 110 and/or the reaction tube 120.

In some embodiments, a side wall of the tube holder 132 may be provided with at least one notch 1321, and the uniform mixing mechanism 130 may further comprise a position monitoring component 133. The position monitoring component 133 is configured to monitor a rotational position of the tube holder 132, such that when the tube holder 132 stops rotating, the at least one notch 1321 faces the corresponding magnet 141 so as to perform magnetic attraction processing on the biological sample in the sample tube 110 and/or the reaction tube 120. By providing the structure, in which the notch 1321 of the tube holder 132 is matched with the position monitoring component 133, in the uniform mixing mechanism 130, the rotational position of the notch 1321 during the uniform mixing processing can be conveniently monitored, such that when the notch 1321 rotates to a position close to the magnet 141, the uniform mixing mechanism 130 is controlled to stop rotating. Such an arrangement satisfies magnetic attraction positioning control in various uniform mixing processing control processes and ensures the accuracy of magnetic attraction positioning, thereby improving the uniform mixing processing efficiency and the magnetic attraction processing efficiency of the sample processing apparatus and the automatic extraction device thereof.

In some embodiments, the corresponding tube holder 132 of the sample tube 110 and/or the reaction tube 120 may be provided with one notch on one side of the tube holder 132, as long as the notch can allow for the magnetic attraction processing of the sample tube 110 and/or the reaction tube 120. The embodiments of the present application do not specifically limit the position where the notch is provided. In some embodiments, in order to implement multi-directional and convenient magnetic attraction processing operations on the sample tube 110 and/or the reaction tube 120, the corresponding tube holder 132 of the sample tube 110 and/or the reaction tube 120 may be provided with at least two notches 1321 on at least two sides of the tube holder 132, for example, one notch 1321 is provided on each of two opposite sides of the tube holder 132. In some embodiments, the opening area of the notch 1321 may be greater than the magnetic attraction action area of the magnetic attraction mechanism 140. For example, the opening area of the notch 1321 may be greater than the area of the magnetic attraction surface of the magnet 141 facing the notch 1321.

In some embodiments, the position monitoring component 133 may comprise a position sensor. In some embodiments, the position sensor may be at least one of the following types of sensors: a photoelectric sensor, a Hall sensor, and a laser sensor.

In some embodiments, the position monitoring component 133 may comprise a photoelectric sensor 1331 and a baffle 1332. The photoelectric sensor 1331 is fixed relative to a housing of the uniform mixing driving motor 131, the baffle 1332 is fixed relative to the corresponding tube holder 132, and the photoelectric sensor 1331 is configured to detect the position of the baffle 1332 and thus detect the rotational position of the corresponding tube holder 132. In some embodiments, the baffle 1332 is arranged corresponding to the notch 1321 of the tube holder 132 in the Z-axis direction (i.e., the third direction). In some embodiments, when the tube holder 132 drives the baffle 1332 to rotate, the rotational position of the tube holder 132 may be determined according to photoelectric signal changes monitored by the photoelectric sensors 1331 and a corresponding monitoring program control, so as to determine the position of the notch 1321, and the rotation or stop of the tube holder 132 is then controlled accordingly by determining whether the position of the notch 1321 faces the magnet 141. For example, when it is monitored that the opening position of the notch 1321 faces the magnets 141, the tube holder 132 is controlled to stop rotating so as to perform the magnetic attraction processing. In some embodiments, a monitoring program comprises a corresponding mapping relationship of the baffle 1332, the tube holder 132, the notch 1321, and/or the magnet 141, for example, a corresponding mapping relationship between the baffle 1332 and the notch 1321. In some embodiments, the photoelectric sensor 1331 may be an optic coupling sensor, and the baffle 1332 may be an optic coupling baffle. The use of an optic coupling component with a higher light sensing sensitivity can further improve the accuracy of position monitoring of the notch.

In some embodiments, the magnet 141 of the magnetic attraction mechanism 140 can cover a plurality of processing channels so as to perform magnetic attraction processing on the biological samples in the plurality of sample tubes 110 and/or the biological samples in the plurality of reaction tubes 120 of the plurality of processing channels, so that the magnetic attraction processing of the plurality of processing channels can be implemented by using only a single magnet 141, the mounting of the apparatus is simple and convenient, and operations are convenient and fast. In some embodiments, the magnetic attraction mechanism 140 may comprise a plurality of magnets 141, and each magnet 141 is configured to perform the magnetic attraction processing on the biological sample in the sample tube 110 and/or the biological sample in the reaction tube 120 in one processing channel, so as to facilitate separate control of the magnetic attraction processing on different processing channels, and meet the personalized demands for different processing channels. Moreover, a single magnet (e.g., magnetic iron) is provided in a single processing channel, thereby reducing the production cost of the magnet. In some embodiments, each sample tube 110 or each reaction tube 120 and the corresponding magnet 141 thereof are respectively provided with a position monitoring component 133, so as to implement the position monitoring of each sample tube 110 or each reaction tube 120 and the separate control of the magnetic attraction processing thereof.

In some embodiments, the magnetic attraction mechanism 140 may comprise a first magnetic attraction mechanism 142. The first magnetic attraction mechanism 142 may comprise a first magnet 1421 and a first magnetic attraction driving motor 1422. The first magnet 1421 is in transmission connection with the first magnetic attraction driving motor 1422. The first magnet 1421 is provided between the sample tube 110 and the reaction tube 120. The first magnetic attraction driving motor 1422 can drive the first magnet 1421 to move between the sample tube 110 and the reaction tube 120 so as to perform the magnetic attraction processing on the biological sample in the sample tube 110 and/or the biological sample in the reaction tube 120. For example, the first magnetic attraction driving motor 1422 can drive the first magnet 1421 to move between the sample tube 110 and the reaction tube 120, for example, in the X-axis direction (i.e., the second direction) and/or the Y-axis direction (i.e., the first direction), causing the first magnet 1421 to approach the sample tube 110 and/or the reaction tube 120. In some embodiments, the first magnet 1421 may be provided on the other side of the sample tube 110 away from the reaction tube 120 in the X-axis direction.

In some embodiments, the first magnetic attraction mechanism 142 may further comprise a magnetic shield 1423. The magnetic shield 1423 is provided between an upper portion of the first magnet 1421 and the reaction tube 120, so as to adapt to the situation that there is a small amount of biological sample in the reaction tube 120, which is closer to the bottom, thereby implementing more effective corresponding magnetic attraction processing. In some embodiments, the magnetic shield 1423 may be a magnetic shielding plate made of pure iron, an iron-nickel alloy, etc.

In some embodiments, the first magnet 1421 may comprise an upper-layer sub-magnet 1421a and a lower-layer sub-magnet 1421 b arranged in the vertical direction (Z-axis direction (i.e., the third direction)). The magnetic shield 1423 is provided between the upper-layer sub-magnet 1421a and the reaction tube 120, and likewise implements the control of the magnetic attraction action area during the magnetic attraction processing, so as to adapt to the magnetic attraction requirements of the reaction tube 120 more efficiently.

In some embodiments, the first magnetic attraction mechanism 142 may comprise a second magnetic attraction driving motor 1424. The second magnetic attraction driving motor 1424 is configured to drive the first magnet 1421 to move in the vertical direction (Z-axis direction (i.e., the third direction)), so as to achieve the separate control of the first magnetic attraction mechanism 142 in the vertical direction, further optimizing a movement control function of the first magnetic attraction mechanism 142, and adapting to a more complex working scenario for magnetic attraction processing (e.g., enabling the first magnet 1421 to perform the magnetic attraction processing on only the lower portion of the reaction tube).

In some embodiments, the first magnetic attraction mechanism 142 may comprise a first magnetic attraction bracket 1425 arranged in the Y-axis direction (i.e., the first direction). In some embodiments, two ends of the first magnetic attraction bracket 1425 may be respectively provided on two linear guide rails 1426 arranged in the X-axis direction (i.e., the second direction), so as to achieve better positioning and guiding. In some embodiments, the first magnetic attraction driving motor 1422 is connected to the first magnetic attraction bracket 1425, so as to drive the first magnetic attraction mechanism 142 to move in the X-axis direction (i.e., the second direction). A lead screw 1427 is provided between the first magnetic attraction driving motor 1422 and the first magnetic attraction bracket 1425 in the X-axis direction (i.e., the second direction). In some embodiments, the first magnetic attraction driving motor 1422 may be a lead screw motor or any other possible types of motors. In some embodiments, the first magnetic attraction driving motor 1422 may drive the first magnetic attraction mechanism 142 to move in the X-axis direction (i.e., the second direction) by means of a lead screw-nut mechanism.

In some embodiments, the magnetic attraction mechanism 140 may further comprise a second magnetic attraction mechanism 143. The second magnetic attraction mechanism 143 may comprise a second magnet 1431 and a third magnetic attraction driving motor 1432. The second magnet 1431 is in transmission connection with the third magnetic attraction driving motor 1432. The second magnet 1431 and the first magnet 1421 are provided on two opposite sides of the reaction tube 120. The third magnetic attraction driving motor 1432 can drive the second magnet 1431 to move relative to the reaction tube 120, for example, move in the X-axis direction (i.e., the second direction) and/or the Y-axis direction (i.e., the first direction), so as to perform magnetic attraction processing on the biological sample in the reaction tube 120. Since the first magnetic attraction mechanism 142 and the first magnet 1421 thereof, and the second magnetic attraction mechanism 143 and the second magnet 1431 thereof are respectively arranged corresponding to the respective sample tube 110 and the respective reaction tube 120, it is possible to further ensure respective good magnetic attraction effects of the sample tube 110 and the reaction tube 120, and also implement the separate magnetic attraction processing controls of the sample tube and the reaction tube, meeting the higher requirements for the magnetic attraction processing during sample processing, and improving the overall sample processing efficiency.

In some embodiments, the magnetic attraction action area of the second magnet 1431 may be different from the magnetic attraction action area of the first magnet 1421. In some embodiments, the magnetic attraction action area of the second magnet 1431 may be the same as the magnetic attraction action area of the first magnet 1421.

In some embodiments, the magnetic attraction action area of the second magnet 1431 is less than that of the first magnet 1421. As the second magnet 1431 approaches the reaction tube 120, the second magnet 1431 approaches the bottom of the reaction tube 120. By means of the separate magnetic attraction control of the second magnet 1431 of the second magnetic attraction mechanism 143, the magnetic attraction requirements of the reaction tube 120 are met in a targeted manner.

In some embodiments, the second magnetic attraction mechanism 143 may further comprise a fourth magnetic attraction driving motor 1433. The fourth magnetic attraction driving motor 1433 is configured to drive the second magnet 1431 to move in the vertical direction (Z-axis direction (i.e., the third direction)), so as to achieve separate control of the second magnetic attraction mechanism 143 in the vertical direction.

In some embodiments, the second magnetic attraction mechanism 143 may comprise a second magnetic attraction bracket 1434 arranged in the Y-axis direction (i.e., the first direction). In some embodiments, two ends of the second magnetic attraction bracket 1434 may be respectively provided on two linear guide rails 1426 arranged in the X-axis direction (i.e., the second direction), so as to achieve better positioning and guiding. In some embodiments, the third magnetic attraction driving motor 1432 is connected to the second magnetic attraction bracket 1434, so as to drive the second magnetic attraction mechanism 143 to move in the X-axis direction (i.e., the second direction). One or more guiding shafts 1435 are provided between the third magnetic attraction driving motor 1432 and the second magnetic attraction bracket 1434 in the X-axis direction (i.e., the second direction). In some embodiments, the third magnetic attraction driving motor 1432 may be a lead screw motor or any other possible types of motors.

In some embodiments, when a plurality of processing channels are provided, the first magnetic attraction mechanism 142 may comprise a plurality of first magnets 1421, the second magnetic attraction mechanism 143 may comprise a plurality of second magnets 1431, and the plurality of first magnets 1421 and the plurality of second magnets 1431 correspond to the plurality of sample tubes 110 and reaction tubes 120 on a one-to-one basis. In some embodiments, the first magnetic attraction mechanism 142 may comprise one first magnetic attraction driving motor 1422 and one second magnetic attraction driving motor 1424; and the second magnetic attraction mechanism 143 may comprise one third magnetic attraction driving motor 1432 and one fourth magnetic attraction driving motor 1433. In some embodiments, the first magnetic attraction mechanism 142 may comprise a plurality of first magnetic attraction driving motors 1422 and a plurality of second magnetic attraction driving motors 1424; and the second magnetic attraction mechanism 143 may comprise a plurality of third magnetic attraction driving motors 1432 and a plurality of fourth magnetic attraction driving motors 1433. In some embodiments, the magnetic attraction processing of the second magnet 1431 and the first magnet 1421 between the processing channels can meet predetermined magnetic attraction requirements (such as stronger magnetic attraction action, or weaker magnetic attraction action), without interfering with each other. With regard to the corresponding sample tube 110 and reaction tube 120 of each of the plurality of processing channels, the first magnetic attraction mechanism 142 and the second magnetic attraction mechanism 143 are respectively provided with the corresponding first magnet 1421, the corresponding second magnet 1431 and the corresponding magnetic attraction driving motor, such that the requirements for respective separate magnetic attraction processing can be met, the driving movement control during the respective separate magnetic attraction processing can also be facilitated (especially suitable for differential magnetic attraction processing between different processing channels and complex sample processing scenarios having differential motor driving program settings), thereby improving the overall processing efficiency of the sample processing apparatus 100.

In some embodiments, the sample processing apparatus 100 may further comprise a support structure 150 for implementing good support for the sample tube 110, the reaction tube 120, the uniform mixing mechanism 130 and the magnetic attraction mechanism 140, thereby improving the stability of the apparatus while ensuring the flexibility of overall translation.

In some embodiments, the automatic extraction device may comprise one or more extraction channels. For example, one or more (e.g., 3, 6, or 8) extraction channels may be provided according to the number of samples to be processed at a time. Each extraction channel may comprise one sample tube, one or more (e.g., 1, 2, or 3) reaction tubes, one reagent kit, one pipette tip box, and one pipette. In some embodiments, each extraction channel can be covered by an external magnetic field for performing magnetic attraction processing on the biological sample in the sample tube and/or the reaction tube of each channel. For example, the external magnetic field may be generated by a single magnet capable of covering a plurality of extraction channels. For another example, each extraction channel corresponds to one magnet, and the external magnetic field can be generated by the corresponding magnet of each channel. By providing the plurality of extraction channels, each extraction channel may process a biological sample, and the extraction processes of the plurality of extraction channels may be performed simultaneously, so that the efficiency of biological sample processing is improved.

FIG. 18 is a schematic diagram of a plurality of extraction channels of an automatic extraction device according to some embodiments of this specification. As shown in FIG. 18, the automatic extraction device 1800 may comprise a plurality of (e.g., 6) extraction channels 1810. A single extraction channel 1810 may comprise one sample tube 1811, a magnet 1812, one reaction tube 1813, a magnet 1814, one reagent kit 1815, one pipette (not shown), and one pipette tip box (not shown). The plurality of extraction channels 1810 are arranged at intervals in the Y-axis direction (i.e., the first direction). Each assembly of a single extraction channel 1810 extends in the X-axis direction (i.e., the second direction). A plurality of sample tubes 1811, a plurality of reaction tubes 1813, a plurality of reagent kits 1815, a plurality of pipettes, and a plurality of pipette tip boxes are respectively aligned in the Y-axis direction (i.e., the first direction). In some embodiments, a plurality of magnets 1812 or a plurality of magnets 1814 may be replaced by a single magnet or a plurality of stacked magnets capable of covering the plurality of extraction channels 1810. In some embodiments, the plurality of magnets 1814 may be omitted. In some embodiments, a single extraction channel 1810 may comprise one waste liquid box (not shown).

In some embodiments, the automatic extraction device (shown in FIGS. 1-3 and/or FIG. 18) described in the specification of the present application can be applied to extracting and/or purifying a target biological substance. A use method for the automatic extraction device described in this specification will be provided to extract, separate and/or purify a target biological substance. Before the automatic extraction device is started, the pipette tip box is loaded on the consumable supporting seat, the reagent kit is loaded on the reagent kit holder and then the entire reagent kit holder is loaded on the consumable supporting seat, and the sample tube and the reaction tube each containing a biological sample are loaded the tube holder. After loading is completed, the automatic extraction device may be started to automatically extract and/or purify the target biological substance.

In some embodiments, the use method for the automatic extraction device may comprises the following steps.

In step 1, the pipetting apparatus is controlled to add a first reagent and a first magnetic bead of the consumable assembly into the sample tube of the sample processing apparatus.

In some embodiments, the first magnetic bead is configured to bind to impurities or the target biological substance. In some embodiments, the first magnetic bead and the first reagent may be a mixture contained in one cavity of the reagent kit.

In step 2, the uniform mixing mechanism is controlled to drive the sample tube for uniform mixing processing.

By means of the uniform mixing processing, the biological sample and the reagent in the sample tube can fully react with each other, and/or the first magnetic bead can fully bind to the impurities or the target biological substance.

In some embodiments, the start and stop of the uniform mixing process and the rotation speed and the rotation direction of the sample tube are controlled by controlling the uniform mixing driving motor.

In step 3, the magnetic attraction mechanism is controlled to perform magnetic attraction processing on a uniformly mixed liquid in the sample tube.

In some embodiments, the first magnet may be controlled to move closer to the sample tube by controlling the first magnetic attraction driving motor, so as to obtain a better magnetic attraction effect. In some embodiments, the second magnetic attraction driving motor may be controlled to drive the first magnet to move up and down according to the amount of a solution in the sample tube, so as to better attract a first magnetic bead conjugate in the sample tube.

In step 4, after standing for a first preset time, the pipetting apparatus is controlled to collect a first supernatant from the sample tube, or to remove the first supernatant from the sample tube while retaining the attracted first magnetic bead.

In some embodiments, the first preset time may be a time during which the automatic extraction device stops operating. In some embodiments, the first preset time may be manually set according to extraction and purification processes or be set by default in the automatic extraction device. In some embodiments, within the first preset time, the magnetic attraction mechanism maintains magnetic attraction to the uniformly mixed liquid in the sample tube.

The supernatant here refers to a transparent liquid above a sedimentation layer (e.g., the first magnetic bead conjugate) in the sample tube. Taking an example that the first magnetic bead is configured to bind to the impurities, the first supernatant mainly contains the target biological substance to be extracted and a small amount of impurities. The use method for the automatic extraction device may further comprises the following steps.

In step 5, the pipetting apparatus is controlled to add the first supernatant from the sample tube into the reaction tube, and the pipetting apparatus is controlled to add a second reagent and a second magnetic bead of the consumable assembly into the reaction tube.

In some embodiments, the second reagent is the same as the first reagent, and/or the first magnetic bead is the same as the second magnetic bead. For example, objects to be bound by the second magnetic bead and the first magnetic bead are consistent. For example, both the first magnetic bead and the second magnetic bead are configured to bind to impurities. As another example, the second magnetic bead may be the same as or different from the first magnetic bead. In some embodiments, the second reagent is different from the first reagent, and/or the second magnetic bead is different from the first magnetic bead. For example, the first magnetic bead is configured to bind to the impurities, and the second magnetic bead is configured to bind to the target biological substance. In some embodiments, the second reagent and the second magnetic bead may be a mixture contained in one cavity of the reagent kit.

In step 6, the uniform mixing mechanism is controlled to drive the reaction tube for uniform mixing processing.

In some embodiments, the start and stop of the uniform mixing process and the rotation speed and the rotation direction of the sample tube are controlled by controlling the uniform mixing driving motor.

In step 7, the magnetic attraction mechanism is controlled to perform magnetic attraction processing on the uniformly mixed liquid in the reaction tube.

In some embodiments, the third magnetic attraction driving motor may be controlled to drive the second magnet to move closer to the reaction tube, so as to obtain a better magnetic attraction effect. In some embodiments, the second magnetic attraction driving motor may be controlled to drive the first magnet to move up and down, so as to better attract a second magnetic bead conjugate in the sample tube.

In step 8, after standing for a second preset time, the pipetting apparatus is controlled to remove a second supernatant from the reaction tube while retaining the attracted second magnetic bead, or to collect the second supernatant from the reaction tube is collected.

In some embodiments, the second preset time may be a time during which the automatic extraction device stops operating. In some embodiments, the second preset time may be manually set according to extraction and purification processes or be set by default in the automatic extraction device. In some embodiments, within the second preset time, the magnetic attraction mechanism maintains magnetic attraction to the uniformly mixed liquid in the reaction tube.

In some embodiments, when the second magnetic bead is configured to bind to the impurities, the pipetting apparatus is controlled to collect the second supernatant from the reaction tube, which is the target biological substance to be pipetted and/or purified.

In some embodiments, when the second magnetic bead is configured to bind to the target biological substance, the pipetting apparatus is controlled to remove the second supernatant from the reaction tube. The second supernatant in the reaction tube is a waste liquid. In this case, the method may proceed to step 9. Step 9 comprises the following sub-steps.

In sub-step 9a, after the magnetic attraction mechanism is controlled to stop the magnetic attraction, the pipetting apparatus is controlled to add a third reagent of the consumable assembly into the reaction tube, the third reagent being capable of eluting the target biological substance from the second magnetic bead.

In sub-step 9b, the uniform mixing mechanism is controlled to drive the reaction tube for uniform mixing processing.

In sub-step 9c, the magnetic attraction mechanism is controlled to perform magnetic attraction processing on the uniformly mixed liquid in the reaction tube.

In sub-step 9d, after standing for a third preset time, the pipetting apparatus is controlled to collect a third supernatant from the reaction tube.

The third supernatant is the target biological substance to be pipetted and/or purified. In some embodiments, the third preset time may be a time during which the automatic extraction device stops operating. In some embodiments, the third preset time may be manually set according to extraction and purification processes or be set by default in the automatic extraction device. In some embodiments, within the third preset time, the magnetic attraction mechanism maintains magnetic attraction to the uniformly mixed liquid in the reaction tube.

In some embodiments, steps 5-8 may be repeated one or more times (e.g., twice) before sub-step 9a, so as to sufficiently remove the impurities by means of the waste liquid.

In some embodiments, steps 1 and 5 comprise a liquid aspiration operation and a liquid dispensing operation. When the liquid aspiration operation is performed, the liquid aspiration step comprises: after assembling the pipette tip, controlling the moving beam driving motor to move the moving beam in the X-axis direction (i.e., the second direction) to a position where the liquid aspiration operation needs to be performed, such that the pipette is located above the position; controlling the pipetting apparatus driving motor to move the pipette down in the Z-axis direction (i.e., the third direction) until the pipette tip is in contact with the solution; and controlling the piston driving motor to drive the piston to move up, so as to implement the liquid aspiration operation. In some embodiments, assembling the pipette tip may comprise: controlling the moving beam driving motor to move the moving beam to the pipette tip box, such that the pipette is located above the pipette tip to be assembled; controlling the pipetting apparatus driving motor to move the pipette down in the Z-axis direction (i.e., the third direction) until the suction head comes into contact with and is sealingly connected to the pipette tip; and moving the pipette up in the Z-axis direction (i.e., the third direction) to remove the pipette tip from the pipette tip box. In some embodiments, assembling the pipette tip may comprise: after the pipette is located above the pipette tip, controlling the pipetting apparatus driving motor to move the pipette down in the third direction to reach an appropriate position; and controlling the piston driving motor to cause the driving plate to push the push rod so as to move the push plate down until the suction head comes into contact with and is sealingly connected to the pipette tip. The spring provided on the push rod can act as a buffer for the piston driving motor, to reduce the impact of the piston driving motor onto the suction head, such that violent collision between the suction head and the pipette tip can be avoided, and thus damage to the suction head and/or the pipette tip can be avoided. In some embodiments, the position of the pipette tip box relative to the base 201 may be adjusted in the Y-axis direction (i.e., the first direction) by controlling the consumable assembly driving motor, so as to align the pipette tip in the pipette tip box with the pipette.

In some embodiments, when the liquid aspiration operation is performed, a piercing step is required before the liquid aspiration step. The piercing step may comprise: controlling the piercing mechanism to move in the third direction, such that the piercing mechanism pierces the sealing layer of the reagent kit. In some embodiments, the piercing step may further comprise: controlling the moving beam driving motor to move the moving beam to the reagent kit, such that the pipette is located above the reagent kit; controlling the pipetting apparatus driving motor to move the pipette down to reach an appropriate position in the Z-axis direction (i.e., the third direction); and controlling the piston driving motor to cause the driving plate to push the push rod so as to move the push plate down until the piercing mechanism pierces the sealing layer, such that the pipette tip can pass through a pierced hole in the sealing layer to aspirate the reagent. In some embodiments, the position of the reagent kit relative to the base 201 may be adjusted in the Y-axis direction (i.e., the first direction) by controlling the consumable assembly driving motor, so as to align the cavity of the reagent kit with the pipette.

In some embodiments, when the liquid dispensing operation is performed, the liquid dispensing step comprises: controlling the moving beam driving motor to move the moving beam to a position where the liquid dispensing operation needs to be performed, such that the pipette is located above the position; controlling the pipetting apparatus driving motor to move the pipette down in the Z-axis direction (i.e., the third direction) until the pipette tip is in contact with the solution; and controlling the piston driving motor to drive the piston to move down, so as to implement the liquid dispensing operation. The detailed steps of the liquid dispensing operation are similar to those of the liquid aspiration operation and will not be repeated here.

Taking an example that the first magnetic bead is configured to bind to the target biological substance, the first supernatant is the waste liquid. The use method for the automatic extraction device further comprises: after controlling the pipetting apparatus to remove the first supernatant from the sample tube while retaining the attracted first magnetic bead, controlling the magnetic attraction mechanism to stop the magnetic attraction; controlling the pipetting apparatus to add a fourth reagent of the consumable assembly into the sample tube for eluting the target biological substance from the first magnetic bead; controlling the uniform mixing mechanism to drive the sample tube for uniform mixing processing; controlling the magnetic attraction mechanism to perform magnetic attraction processing on the uniformly mixed liquid in the sample tube; and controlling the pipetting apparatus to collect a fourth supernatant from the sample tube, which is the target biological substance to be extracted and/or purified.

The possible beneficial effects of the embodiments of this specification include but are not limited to the following aspects: (1) by providing the pipetting apparatus, the consumable assembly and the sample processing apparatus on the portal frame, the operator can automatically operate the automatic extraction device only by loading the consumables and the biological samples into the automatic extraction device, thereby implementing the mechanical automation of extracting a target biological substance, and saving a lot of labor costs; (2) by providing the plurality of extraction channels in parallel for the automatic extraction device, a plurality of biological samples can be processed simultaneously by means of the plurality of extraction channels, such that the efficiency of biological sample processing is improved, and the extraction quality and yield of the target biological substance are also improved; (3) by means of the automatic mechanical control of the uniform mixing mechanism and the magnetic attraction mechanism in the sample processing apparatus, the mechanical operations of uniform mixing processing and magnetic attraction processing during the biological sample processing are implemented, and the operation efficiency is improved; (4) by controlling the movements of the pipetting apparatus in multiple directions with the pipetting apparatus driving motor and the piston driving motor, the mechanical pipetting operation during the biological sample processing is implemented, and the pipetting operation requirements at different stages during the biological sample processing can be met, thereby improving the mechanization of the automatic extraction device; (5) by providing the piercing mechanism in the pipetting apparatus, the sealing layer of the reagent kit can be mechanically pierced before the liquid is aspirated from the reagent kit, and the automation of the liquid pipetting operation and the biological sample processing is improved; (6) by controlling the movement of the consumable assembly with the consumable driving motor, it is applicable to situations where there are many cavities in the reagent kit and/or there are many pipette tips, and the automatic extraction device can be not too long in a channel direction due to the use of the multi-row arrangement, so that the automatic extraction device can be configured reasonably in structure; (7) by loading the plurality of reagent kits on the reagent kit holder, it is convenient for the operator to integrally load/unload the plurality of reagent kits to/from the automatic extraction device, thereby improving the operation convenience of the automatic extraction device; and (8) by controlling the movements of the uniform mixing mechanism and the magnetic attraction mechanism in multiple directions with the uniform mixing driving motor and the magnetic attraction driving motor, the automatic operation efficiency of the sample processing apparatus and the automatic extraction device is further improved, and a variety of demands for the uniform mixing processing and the magnetic attraction operation can be met. It should be noted that different embodiments may have different beneficial effects, and the beneficial effects that may be produced in the different embodiments may be any one or a combination of the above, or may be any other beneficial effects that may be obtained.

The basic concepts have been described above, and it will be apparent to those skilled in the art that the foregoing detailed disclosure is intended to be exemplary only and does not constitute a limitation to this specification. Although not explicitly stated herein, those skilled in the art may make various modifications, improvements and amendments to this specification. Such modifications, improvements and amendments are suggested in this specification, and thus remain within the spirit and scope of the exemplary embodiments of this specification.

Meanwhile, this specification uses specific words to describe the embodiments of this specification. For example, “one embodiment”, “an embodiment” and/or “some embodiments” mean a particular feature, structure or characteristic related to at least one embodiment of this specification. Hence, it should be emphasized and noted that “an embodiment” or “one embodiment” or “one alternative embodiment” mentioned in two or more different positions in this specification does not necessarily refer to the same embodiment. Furthermore, some features, structures or characteristics in one or more embodiments of this specification may be appropriately combined.

Furthermore, unless explicitly stated in the claims, the order of processing elements and sequences, the use of numbers and letters, or the use of other names described in this specification is not intended to limit the order of the process or method of this specification. Although some currently considered useful embodiments have been discussed in the above disclosure by way of various examples, it should be understood that such details are only for illustrative purposes, and that the appended claims are not limited to the disclosed embodiments; rather, the claims are intended to cover all amendments and equivalent combinations that are in line with the spirit and scope of the embodiments of this specification. For example, although the system components described above can be implemented by means of hardware devices, they may also be implemented only by software solutions, such as installing the described system on an existing server or mobile device.

Similarly, it should be noted that in order to simplify the presentation of the disclosure of this specification, and thereby help to understand one or more embodiments, in the previous descriptions of the embodiments of this specification, various features are sometimes combined into one embodiment, the accompanying drawings, or descriptions thereof. However, this method of disclosure does not imply that the subject of this specification requires more features than those mentioned in the claims. In fact, the embodiments have fewer features than all of the individual embodiments disclosed above.

In some embodiments, numbers for describing components, properties and quantities are used. It should be understood that such numbers for describing the embodiments are modified by the modifiers “about”, “approximately” or “substantially” in some examples. Unless otherwise stated, the modifiers “about”, “approximately” or “substantially” indicate that a number is allowed to vary by ±20%. Accordingly, in some embodiments, numerical parameters used in the specification and claims are approximate values that may vary depending on desired features of individual embodiments. In some embodiments, with regard to the numerical parameters, the specified number of significant digits should be taken into account, and a general digit retaining method should be used. Although numerical ranges and parameters for determining the breadth of ranges in some embodiments of this specification are approximate values, such numerical values should be set as accurate as possible in feasible ranges in the specific embodiments.

Each patent, each patent application, each patent application publication and other materials, such as articles, books, instructions, publications, documents, etc. cited in this specification are hereby incorporated by reference into this specification in its entirety. Application history documents that are inconsistent with or conflict with the contents of this specification are excluded, and documents (currently or later appended to this specification) that limit the broadest scope of the claims of this specification are also excluded. It should be noted that if there is any inconsistency or conflict between the descriptions, definitions, and/or the use of terms in the accompanying materials of this specification and the contents of this specification, the descriptions, definitions, and/or the use of terms in this specification shall prevail.

Finally, it should be understood that the embodiments described in this specification are only intended to illustrate the principles of the embodiments of this specification. Other variations may also fall within the scope of this specification. Thus, by way of example but not limitation, alternative configurations of the embodiments of this specification can be considered consistent with the teachings of this specification. Accordingly, the embodiments of this specification are not limited to those explicitly presented and described in this specification.

1. An automatic extraction device for extracting a target biological substance, the automatic extraction device comprising: a sample processing apparatus, a consumable assembly, a pipetting apparatus and a portal frame, wherein
the sample processing apparatus comprises a uniform mixing mechanism and a magnetic attraction mechanism, which are respectively configured to perform uniform mixing processing and magnetic attraction processing on a biological sample;
the portal frame comprises a base and a moving beam capable of moving relative to the base; and
the sample processing apparatus and the consumable assembly are provided on the base, and the pipetting apparatus is provided on the moving beam and configured to be capable of implementing a pipetting operation between the consumable assembly and the sample processing apparatus.

2. The automatic extraction device of claim 1, wherein the sample processing apparatus comprises at least one of a sample tube and a reaction tube; and
the magnetic attraction mechanism comprises a magnet capable of moving relative to the sample tube and/or the reaction tube so as to perform magnetic attraction processing on a biological sample in the sample tube and/or a biological sample in the reaction tube.

3. The automatic extraction device of claim 1, wherein the uniform mixing mechanism comprises a uniform mixing driving motor and a tube holder, the uniform mixing driving motor being in transmission connection with the tube holder;
the sample tube and/or the reaction tube are provided in the tube holder; and
the uniform mixing driving motor is capable of driving the tube holder to move, so as to drive the biological sample in the sample tube and/or the biological sample in the reaction tube to undergo uniform mixing processing,
wherein the tube holder is connected to a rotating shaft of the uniform mixing driving motor, and the uniform mixing driving motor is capable of driving the tube holder to rotate axially.

4. (canceled)

5. The automatic extraction device of claim 2, wherein the magnetic attraction mechanism comprises a first magnetic attraction mechanism, the first magnetic attraction mechanism comprising a first magnet and a first magnetic attraction driving motor, wherein the first magnet is in transmission connection with the first magnetic attraction driving motor;
the first magnet is provided between the sample tube and the reaction tube; and
the first magnetic attraction driving motor is capable of driving the first magnet to move between the sample tube and the reaction tube, so as to perform magnetic attraction processing on the biological sample in the sample tube or in the reaction tube,
wherein the first magnetic attraction mechanism further comprises a second magnetic attraction driving motor; and the second magnetic attraction driving motor is configured to drive the first magnet to move in a vertical direction.

6. (canceled)

7. The automatic extraction device of claim 5, wherein the magnetic attraction mechanism further comprises a second magnetic attraction mechanism, the second magnetic attraction mechanism comprising a second magnet and a third magnetic attraction driving motor, wherein the second magnet is in transmission connection with the third magnetic attraction driving motor;
the second magnet and the first magnet are provided on two opposite sides of the reaction tube; and
the third magnetic attraction driving motor is capable of driving the second magnet to move relative to the reaction tube, so as to perform magnetic attraction processing on the biological sample in the reaction tube.

8. The automatic extraction device of claim 1, wherein the consumable assembly comprises a reagent kit and a pipette tip box, the reagent kit comprising one or more cavities for containing reagents, and the pipette tip box comprising one or more pipette tips; and
the pipetting apparatus comprises a pipette capable of being detachably and sealingly connected to the pipette tip, and when the pipette is sealingly connected to the pipette tip, the pipette is capable of controlling the pipette tip to implement liquid aspiration and dispensing,
wherein the pipette tip box corresponds to the reagent kit on a one-to-one basis.

9. (canceled)

10. The automatic extraction device of claim 8, wherein the reagent kit comprises a plurality of cavities, and the pipette tip box comprises a plurality of pipette tips;
the plurality of cavities are arranged in one or more rows in a first direction, each row of cavities extends in a second direction in which the moving beam moves relative to the base, and the first direction is perpendicular to the second direction; and
the plurality of pipette tips are arranged in one or more rows in the first direction, and each row of pipette tips extends in the second direction.

11. The automatic extraction device of claim 10, further comprising a consumable assembly driving motor configured to drive the consumable assembly to move in the first direction.

12. The automatic extraction device of claim 8, wherein the pipette tip box comprises a box body having an upper end open, and a cover plate, the cover plate being connected to an opening of the box body;
the inside of the box body is divided into a plurality of accommodating cavities by partitions, and the cover plate is provided with a plurality of through holes corresponding to the plurality of accommodating cavities; and
at least some of the accommodating cavities and the through holes corresponding thereto are configured to load the one or more pipette tips,
wherein the pipette tip box further comprises a target biological substance collection tube, at least one of the accommodating cavities and the through hole(s) corresponding thereto are configured to load the target biological substance collection tube.

13. (canceled)

14. The automatic extraction device of claim 8, wherein the reagent kit comprises a sealing layer configured to seal the one or more cavities, wherein the pipetting apparatus further comprises a piercing mechanism configured to pierce the sealing layer.

15. (canceled)

16. The automatic extraction device of claim 1, further comprising a waste liquid box, wherein the waste liquid box is fixed relative to the base; and
the pipetting apparatus is configured to be capable of implementing a pipetting operation between the sample processing apparatus and the waste liquid box.

17. The automatic extraction device of claim 1, wherein the sample processing apparatus comprises a plurality of sample tubes and a plurality of reaction tubes, the consumable assembly comprises a plurality of reagent kits and a plurality of pipette tip boxes, and the pipetting apparatus comprises a plurality of pipettes;
the automatic extraction device comprises a plurality of extraction channels arranged at intervals in a first direction, which is perpendicular to a second direction in which the moving beam moves relative to the base, and each extraction channel extends in the second direction; and
each extraction channel comprises one sample tube, one or more reaction tubes, one reagent kit, one pipette tip box, and one pipette.

18. The automatic extraction device of claim 17, wherein the magnetic attraction mechanism comprises a magnet capable of covering the plurality of extraction channels so as to perform magnetic attraction processing on biological samples in the plurality of sample tubes and/or the plurality of reaction tubes of the plurality of extraction channels.

19. The automatic extraction device of claim 17, wherein the magnetic attraction mechanism comprises a plurality of magnets, each magnet being configured to perform magnetic attraction processing on a biological sample in the sample tube and/or the reaction tube in one extraction channel.

20. The automatic extraction device of claim 1, wherein the biological sample comprises a microorganism, blood, saliva, an animal tissue, a plant, or food, wherein the target biological substance comprises a plasmid, a protein, or a nucleic acid.

21. (canceled)

22. A use method for an automatic extraction device of claim 1, the use method comprising the steps of:
controlling the pipetting apparatus to add a first reagent and a first magnetic bead of the consumable assembly into the sample tube of the sample processing apparatus, with a biological sample being contained in the sample tube;
controlling the uniform mixing mechanism to drive the sample tube for uniform mixing processing;
controlling the magnetic attraction mechanism to perform magnetic attraction processing on a uniformly mixed liquid in the sample tube; after standing for a first preset time, controlling the pipetting apparatus to collect a first supernatant from the sample tube, or to remove the first supernatant from the sample tube while retaining the attracted first magnetic bead and
adding the first supernatant collected from the sample tube into the reaction tube of the sample processing apparatus;
controlling the pipetting apparatus to add a second reagent and a second magnetic bead of the consumable assembly into the reaction tube;
controlling the uniform mixing mechanism to drive the reaction tube for uniform mixing processing;
controlling the magnetic attraction mechanism to perform magnetic attraction processing on the uniformly mixed liquid in the reaction tube; and
after standing for a second preset time, controlling the pipetting apparatus to remove a second supernatant from the reaction tube while retaining the attracted second magnetic bead, or to collect the second supernatant from the reaction tube.

23. (canceled)

24. The use method of claim 22, wherein the first magnetic bead is configured to bind to impurities, and the second magnetic bead is configured to bind to the target biological substance; and the use method further comprises:
after standing for the second preset time, controlling the pipetting apparatus to remove the second supernatant from the reaction tube;
controlling the pipetting apparatus to add a third reagent of the consumable assembly into the reaction tube, the third reagent being capable of eluting the target biological substance from the second magnetic bead;
controlling the uniform mixing mechanism to drive the reaction tube for uniform mixing processing;
controlling the magnetic attraction mechanism to perform magnetic attraction processing on the uniformly mixed liquid in the reaction tube; and
after standing for a third preset time, controlling the pipetting apparatus to collect a third supernatant from the reaction tube.

25. The use method of claim 22, wherein the first magnetic bead and the second magnetic bead are both configured to bind to impurities; and the method further comprises:
after standing for the second preset time, controlling the pipetting apparatus to collect the second supernatant from the reaction tube.

26. The use method of claim 22, wherein the first reagent is the same as the second reagent, and the first magnetic bead is the same as the second magnetic bead.

27. The use method of claim 22, wherein the first reagent is different from the second reagent, and/or the first magnetic bead is different from the second magnetic bead.
